(19) United States
(12) Patent Application Publication
(10) Pub. No.: US 2017/0049941 A1
(43) Pub. Date: Feb. 23, 2017

(54) Method of Preparing Artificial Organs, and Related Compositions

(71) Applicants: University of Pittsburgh-Of the Commonwealth System of Higher Education, Pittsburgh, PA (US); Keio University School of Medicine, Tokyo (JP)

(72) Inventors: Alejandro SOTO-GUTIERREZ, Pittsburgh, PA (US); Kentaro MATSUBARA, Pittsburgh, PA (US); Ken FUKUMITSU, PIttsburgh, PA (US); Kan HANDA, Pittsburgh, PA (US); Jorge Guzman LEPE, Pittsburgh, PA (US); William R. WAGNER, Gibsonia, PA (US); Sang Ho YE, Pittsburgh, PA (US); Hiroshi YAGI, Tokyo (JP); Yuko KITAGAWA, Tokyo (JP)

(21) Appl. No.: 15/307,634

(22) PCT Filed: Apr. 29, 2015
(86) PCT No.: PCT/US2015/028238

§ 371 Date: Oct. 28, 2016

Related U.S. Application Data
(60) Provisional application No. 61/985,690, filed on Apr. 29, 2014.

(51) Int. Cl. A61L 33/06; A61L 33/00; A61L 27/34; A61L 27/36; A61L 27/38

(52) U.S. Cl. CPC A61L 33/068; A61L 27/3687; A61L 27/3633; A61L 27/3804; A61L 27/34; A61L 33/0011; A61L 2300/42

(57) ABSTRACT

Provided herein are methods or making and using whole or partial organ ECM structures comprising an anticoagulant. Also provided are organ structures prepared according to those methods.

### CROSS-REFERENCE TO RELATED APPLICATIONS

This application claims priority to U.S. Provisional Patent Application No. 61/985,690, filed Apr. 29, 2014, the contents of which are incorporated herein by reference in their entirety.

### STATEMENT REGARDING FEDERAL FUNDING

This invention was made with government support under Grant No. DK083556 awarded by the National Institutes of Health. The government has certain rights in the invention.

Financial support for this invention was also provided under the Research Center Network for Realization of Regenerative Medicine, provided by the Japan Agency for Medical Research and Development (AMED).

### BACKGROUND OF THE INVENTION

There is a critical shortage of organs for transplantation, with the organ waiting list currently at over 110,000 requests and increasing by 5% every year. Approximately 30,000 deaths are registered annually in the US due to liver disease. At this time, the only definitive treatment of hepatic failure is orthotopic transplantation. This form of transplantation will always be limited due to a paucity of available organs, and the delivery of cells directly is inefficient due to low engraftment. Thus, the generation of a transplantable tissue engineered liver graft could dramatically change this equation (e.g. organ engineering using natural organs scaffolds). However, the major challenge in tissue/organ engineering (including liver) has so far been limited graft survival after transplantation. The main gap that prevents advancement of the field is the lack of strategies to prevent acute thrombosis after graft transplantation.

Tissue engineering has so far had limited success in many tissues, including liver. The main gap that prevents advancement of the field is the lack of an ideal transplantable scaffold that has all the necessary microstructure and extracellular cues for cell attachment, differentiation, functioning, as well as vascularization, which has so far proven to be difficult to manufacture in vitro. In recent publications in Nature Medicine (Uygun et al. “Organ reengineering through development of a transplantable re-cellularized liver graft using decellularized liver matrix.” (2010) Nat Med. 16(7):814-20) and Tissue Engineering (Soto-Gutierrez et al. “A whole-organ regenerative medicine approach for liver replacement” (2011) Tissue Eng. Part C Methods, 17(6):677-686), cadaveric liver decellularization protocols to create a whole-liver scaffold for engineering hepatic grafts have been demonstrated. The decellularization process preserves intact the native microvascular network of the organ. Adult hepatocytes can be seeded into these scaffolds, remaining viable and providing essential liver functions for up to 10 days. Moreover, engineered livers could be implanted in the rats using the recipient left renal artery and vein. Liver graft function was documented for up to 8 hours after implantation. However, long-term transplantation of engineered livers remains a challenge.

### SUMMARY OF THE INVENTION

Methods of preparing engineered organs with anti-thrombotic activity are provided to achieve long-term survival after transplantation using optimized vascular re-cellularization and/or polymer-based vascular surface modification to block acute thrombosis. The methods provide protocols to mitigate acute thrombosis with reendothelialization and protein-reactive polymers, such as N-hydroxysuccinimide-polyethylene glycol (NHS-PEG) and similar other molecules, and engineered organs for transplantation in patients with impaired organ functionality. Such engineered organs retain vasculature and are suitable for long-term survival following implantation. The organs described herein are based on extracellular matrix (ECM), and can be completely reendothelialized so as to not induce coagulation when exposed to blood (i.e., organs that are not at risk of acute thrombosis).

In one aspect, provided herein is a method of preparing a whole or partial organ extracellular matrix (ECM) construct including the steps of decellularizing a whole organ or partial organ by contacting the whole organ or partial organ with a decellularization solution and coating the decellularized whole organ or partial organ with an anticoagulant protein-associating composition. In some aspects the whole organ or partial organ is a whole liver or partial liver.

In some aspects, the step of decellularizing the whole or partial organ ECM construct includes contacting the whole organ or partial organ with a solution comprising about 0.02% trypsin and then contacting the whole organ or partial organ with a solution comprising about 0.1% Triton X-100.

In some aspects, the whole organ or partial organ is also disinfected. In some aspects, the whole organ or partial organ is disinfected with peracetic acid.

In some aspects of the decellularization solution further includes a chelating agent. In some aspects, the chelating agent is EGTA.

In some aspects of the method of preparing a whole or partial organ ECM construct, the whole organ or partial organ is frozen before decellularization.

In some aspects of the method, the anticoagulant protein-associating composition is a polyether polymer, copolymer, or block copolymer, such as a poly(C1-C6 alkylene oxide) moiety, such as a polyoxyethylene, a polyoxypropylene, or a polyoxytetramethylene linked to an amine or ECM-reactive group. In some aspects, the anticoagulant protein-associating composition includes an N-hydroxysuccinimide (NHS) moiety covalently linked to a non-reactive, hydrophilic, biocompatible polymer moiety. In some aspects of the method of preparing a whole or partial organ ECM construct the biocompatible polymer moiety comprises a polyether polymer, copolymer, or block copolymer, such as a poly(C1-C6 alkylene oxide) moiety, such as a polyoxyethylene, a polyoxypropylene, or a polyoxytetramethylene linked to an amine-reactive group.

In some aspects of the invention, the anticoagulant protein-associating composition includes poly(ethylene glycol) covalently linked to an NHS moiety.

In some aspects of the invention, the protein-associating polymer composition includes a phosphorylcholine (PC), sulfobetaine (SB), or carboxybetaine (CB) moiety.

In some aspects, the anticoagulant protein-associating composition includes an amine or ECM-reactive group. In some aspects the amine or ECM-reactive group is NHS, isocyanate (NCO), or carboxyl (COOH).

In some aspects of the method the anticoagulant protein-associating composition includes one or more of PEG-NHS, PEG-NCO, PC-NHS, PC-NCO, SB-PEG-NHS, PC-COOH, SB-COOH, or poly[N-p-vinylbenzyl-4-O-β-D-galactopyranosyl-D-gluconamide]-co-valine (PVLA-co-VAL).

In some aspects of the method of preparing a whole or partial organ ECM construct, each of the decellularization solution and the anticoagulant protein-associating composition are provided to the whole organ or partial organ by flushing vasculature of the whole organ or partial organ, thereby coating the vasculature with the anticoagulant protein-associating composition.

Also provided herein is a decellularized extracellular matrix (ECM) organ structure. The decellularized ECM organ structure includes a decellularized whole organ or partial organ comprising native ECM structure, and an anticoagulant protein-associating composition dispersed within the native ECM structure.

In some aspects, the anticoagulant protein-associating composition includes one or more of PEG-NHS, PEG-NCO, PC-NHS, PC-NCO, SB-PEG-NHS, PC-COOH, SB-COOH, or PVLA-co-VAL.

In some aspects of the invention, the anticoagulant protein-associating composition includes poly(ethylene glycol) covalently linked to an NHS moiety.

In some aspects of the invention, the organ structure further includes orthotopic, autologous, allogeneic or xenogeneic cells dispersed into the decellularized organ structure. In some aspects, the cells are primary cells, multipotent cells, or pluripotent cells.

### BRIEF DESCRIPTION OF THE DRAWINGS

FIG. 1. Two variations of useful whole organ decellularization protocols suitable for the present invention.

FIG. 2. Optimization and characterization of decellularized rat livers according to an embodiment of the present invention. (a) Representative images of multiphoton microscopy and of normal and decellularized rat livers observed in at least three specimens. (b) SEM images of extracellular matrix within the parenchyma. (c) Glisson's capsule of normal and after liver decellularization. (d) Collagen content of normal and decellularized rat liver using 3% and 0.1% triton X-100 solutions; (e) DNA content of normal and decellularized rat liver using 3% and 0.1% triton X-100 solutions; (f) Comparison of normal liver (top) and decellularized rat liver (bottom). Left to right: fibronectin (red) and laminin (red) staining. Sections were counterstained with DAPI (blue). (g) Thermograms of normal liver (green) and decellularized liver using 3% (blue) and 0.1% (red) triton X-100 solutions.

FIG. 3. Liver graft infusion and perfusion systems suitable for use in the methods of the present invention. Lean decellularized livers can be re-cellularized through portal vein, bile duct and/or inferior vena cava using the cell infusion system. Re-cellularized livers can be successfully culture/perfused at physiological pressures and flow in the organ culture system for long-term periods of time indicating the feasibility of the perfusion system as a graft culture environment.

FIG. 4A-4B. Assembly and function of whole organ vasculature in decellularized rat livers according to an embodiment of the present invention. FIG. 4A: (a) 3D micro-CT angiography of normal and decellularized livers vascular compartments (portal and central vein); (b) Representative micro-MRI images of micron-sized iron oxide particle-labeled endothelial cells seeded into the portal and central vein of decellularized livers; (c) Representative fluorescent confocal microscopy images of the same micron-sized iron oxide particles-labeled endothelial cells assembled portal and central veins of decellularized livers and the corresponding images of histological sections stained with hematoxylin and eosin. Histological quantification of assembled whole organ vasculature is also shown; (d) SEM images of normal liver vasculature and assembled liver vasculature. At least 3 different liver specimens and 3 liver lobes per each were analyzed per group; (e) Representative fluorescence images of assembled liver vasculature (portal and central vein); (f) Vitamin D-stimulated secretion of tissue plasminogen activator (tPA) of microvascular endothelial cells cultured on fibronectin gel, assembled liver vasculature and normal liver; (g) Gene expression analysis of assembled liver vasculature (portal and central vein); FIG. 4B: Imaging and histological quantitative assessment of vascular and bile duct system assembly. (a) Schematic representation of two different types of anatomical remodeling after repopulation of the vascular and bile duct systems using micron-sized iron oxide particle-labeled endothelial cells and cholangiocytes; (b) Schematic representation of the histological quantification of repopulation of bile ducts and vasculature (portal or central vein).

FIG. 5. Assembly of whole organ bile duct in decellularized rat livers according to an embodiment of the present invention. (a) 3D micro-CT angiography of normal and decellularized liver bile duct; (b) Representative micro-MRI images of micron-sized iron oxide particle-labeled cholangiocytes seeded into the bile duct of decellularized livers at different depth levels. Quantification of the liver bile duct repopulation is also shown compared to control paired micro-CT image; (c) Representative fluorescent confocal microscopy images of the same micron-sized iron oxide particle-labeled cholangiocytes assembled bile duct of decellularized livers and the corresponding images of histological sections stained with hematoxylin and eosin. Histological quantification of assembled whole organ bile ducts is also shown; (d) Normalized gene expression of cell-cell and cell-matrix adhesion molecules.

FIG. 6A-6E. Hepatic function and characterization of assembled liver according to an embodiment of the present invention. FIG. 6A: (a) from left to right: urea secretion, albumin synthesis, and total bile acid secretion of assembled liver using combined repopulation protocols; (b) Immunohistochemical staining of the assembled liver compartments (bottom) in comparison to normal liver (top). FIG. 6B: Vascular surface modification of assembled whole livers to prevent early thrombosis according to an embodiment of the present invention. FIG. 6C: Bioengineering of decellularized liver matrix with anti-thrombotic activity using NHS-PEG. (a) Decellularized liver matrix treated with different doses of NHS-PEG-biotin and histological quantification of vessels covered with NHS-PEG-biotin; (b) Representative photographs of NHS-PEG treated decellularized livers and directly perfused with portal blood flow; (c) Immunohistochemical staining for CD41 (platelet marker) and H&E staining of control and NHS-PEG treated decellularized liver matrix after perfusion of portal blood flow. FIG. 6D: (a) Decellularized liver matrix treated with different doses of NHS-PEG-biotin and histological quantification of vessels covered with NHS-PEG-biotin; (b) Representative photographs of NHS-PEG treated decellularized livers and directly perfused with portal blood flow; and (c) Immunohistochemical staining for CD41 (platelet marker) and H&E staining of control and NHS-PEG treated decellularized liver matrix after perfusion of portal blood flow. FIG. 6E: Assembly of liver grafts for transplantation. (a) Liver assembly system for in vitro repopulation of decellularized liver grafts; (b) perfusion chamber with cannulas to access portal vein (PV), inferior vena cava (IVC) and bile duct (BD) for cell delivery; (c) Liver culture system assembled of perfusion chamber, peristaltic pump, oxygenator, bubble trap and access ports; (d) Liver graft assembly protocol.

FIG. 7A-7F. FIG. 7A: (a) Representative images of graft transplantation; left to right: transplant site, transplant site after right nephrectomy, portal vein (PV) preparation for end-to-side anastomosis and auxiliary graft in contrast with the native liver. (b) Representative images of graft transplantation procedure; top, left to right: anterior wall of the infra-hepatic inferior vena cava (IVC) is cut and end-to-side anastomosis is performed, inferior vena cava blood flow is opened, PV is dissected and end-to-side anastomosis is performed; bottom, left to right: IVC and PV are de-clamped and the graft is re-perfused, PV is ligated above the anastomosis, bile duct (BD) of the graft is dissected and inserted into the duodenum. (c) Schematic representation of the auxiliary liver graft transplantation surgical technique for transplantation of normal and assembled liver grafts. (d) Blood rat albumin concentration of normal and assembled liver grafts in liver regeneration-conditioned (retrorsine-treated). FIG. 7B: (a) Representative photographs of gross morphology of an assembled liver graft before and after 17 d of auxiliary liver transplantation in naïve and liver regeneration-conditioned (retrorsine-treated) mutant Nagase analbuminemic rats. (b) Immunohistochemical staining of assembled liver graft after 17 d of auxiliary liver transplantation (bottom two lines) compared to normal liver (top); left to right: albumin (red), Von Willebrand (vW) factor (red), Cytokeratin 19 (CK19) (red) and H&E. FIG. 7C: Infrared image and corresponding photographs of (a) normal and (b) assembled auxiliary liver grafts during transplantation and after 3 weeks of auxiliary liver transplantation. FIG. 7D: Histological analysis of transplanted normal and assembled liver grafts Immunohistochemical staining of normal and assembled liver graft using (a): CYP3A1 (red), (b) Conexxin-32 (Cx32) (red) (a key hepatic gap junction protein) and (c) Integrin beta-1 (ITGB1) (red) (a key transmembrane receptor in the liver). FIG. 7E: Histological analysis of normal and assembled liver grafts after auxiliary liver transplantation. Immunohistochemical staining of normal and assembled liver graft for (a) Collagen type I; and (b) Fibronectin. FIG. 7F: (a) H&E staining of assembled liver graft before transplantation, showing a low and high magnification of the parenchyma space; (b, c) H&E and albumin (red) staining of assembled liver graft 17 d after transplantation in liver regeneration-conditioned (retrorsine-treated) mutant Nagase analbuminemic rats.

FIG. 8. Whole organ porcine liver homogeneous decellularization according to an embodiment of the present invention. Representative images of porcine livers during decellularization process at (a) 0 h; (b) 18 h; (c) 48 h; (d) 72 h; (e) 96 h. (f) DNA was extracted from each different lobe. (g) The DNA content of different lobes of the decellularized liver matrix; and (h) Agarose gel electrophoresis of extracted DNA comparing to that of normal porcine liver. Histologic comparison of normal liver and decellularized liver matrix: (i) hematoxylin and eosin; (j) The presence of intact nuclear material was evaluated by staining the decellularized liver and native liver using 4′,6-diamidino-2-phenylindole (DAPI).

FIG. 9. Establishment of a Model of Hyper-ammonia in the pig by Portacaval shunt. Photographs (superior left) show porta-caval shunt technique and ammonia levels increased over time as shown in the graph. Representative photographs of decellularized livers directly perfused with portal blood flow (center bottom) in pigs to test molecules for anticoagulation according to one embodiment of a liver transplantation model using and testing the methods and organ structures described herein.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values, as well as sub-ranges. For example, a range of temperatures of 4° C. to 37° C. includes 4° C., 5° C., 6° C., 7° C., 8° C., 9° C., 10° C., 15° C., 20° C., 25° C., 30° C., 35° C., 37° C., and sub-ranges such as 15° C. to 20° C. For definitions provided herein, those definitions refer to word forms, cognates and grammatical variants of those words or phrases.

As used herein, the terms “comprising,” “comprise” or “comprised,” and variations thereof, are open ended and do not exclude the presence of other elements not identified. In contrast, the term “consisting of” and variations thereof is intended to be closed, and excludes additional elements in anything but trace amounts.

Provided herein are methods of preparing a whole or partial organ extracellular matrix (ECM) construct that is amenable to full re-endothelialization and suitable for long-term use in transplantation. The method comprises first decellularizing a whole or partial organ, for example a liver or partial liver, followed by providing an anti-thrombic coating to the whole or partial organ. As used herein, a whole or partial organ comprises macro- and micro-level structures such as vasculature, ducts and organ substructures, such as for example, in the case of liver, vasculature and bile ducts. The whole or partial organ is decellularized, leaving behind ECM, but retains organ native structure, meaning that three-dimensional organization of the structures of the organ are substantially retained in the ECM material left behind after decellularization.

One goal of the decellularization protocol is to provide an ECM construct that provides the lowest possibility of an unwanted host response. Parameters suitable for such constructs, such as amount of phospholipid and/or nucleic acid remaining following preparation of the construct, are disclosed, for example, in Keane et al. (“Consequences of ineffective decellularization of biologic scaffolds on the host response” Biomaterials (2012); 1771:1781).

In the experiments described herein, decellularized organ/organ constructs prepared as described herein are used as platforms for organ engineering. For example, although certain decellularization methods of whole or partial organs are known (e.g. as described in U.S. Pat. No. 8,470,520), such decellularized organs do not exhibit sufficient viability in vivo for use in long-term tissue transplantation. Rather, such organs must be re-endothelialized and provided with vascular surface modification proteins with polyethylene glycol (or other like molecules) to inhibit acute thrombosis and achieve long-term graft survival after transplantation. The methods described herein provide a feasible method of preserving the vascular perfusability to allow engineered tissues or organs complete to completely regenerate. As a result of these methods, and whole or partial organs prepared therefrom, long-term survival and regeneration of engineered tissues and organs is possible. The regenerated organs also allow for studying complex liver cell interactions.

For example, engineered liver grafts will enable performing much more aggressive hepatic resections in patients with malignancies, which is currently not possible due to the likelihood of developing hepatic failure as a consequence of insufficient hepatic mass. Moreover, these grafts could be sufficient to support patients with acute liver failure while their own liver recovers, without the risk of performing whole liver transplantation and the use of life-long immunosuppressant therapy. Scientifically, the system provides a feasible model to study liver development and hepatic maturation processes as well as a model to study the complex parenchymal and non-parenchymal liver cell interactions. Engineered organs as described herein could also be used as a tool to accurately predict the metabolism or toxicity of a compound in human liver grafts in vitro prior the exposure to the whole body, by providing a natural environment. This potentially translates into reduced costs and time in drug development, and less harmful patient exposure in clinical trials.

The organ to be decellularized and used as an ECM organ construct may be any organ amenable to decellularization and transplantation. In non-limiting embodiments the organ is a liver, kidney, spleen, gallbladder, lung, heart, muscle, and skin. The organs may be derived from humans, or may be porcine in origin. The organ is decellularized, for example and without limitation, by contacting the whole organ or partial organ by submersion or incubation in a decellularization solution. In a nonlimiting embodiment the decellularization solution is applied to the whole or partial organ by flushing the vasculature (e.g., perfusing) of the organ and/or ductwork of the organ. Decellularization solutions suitable for this use are known to those of skill in the art, but typically are aqueous solutions comprising a detergent or surfactant, and in one embodiment a non-ionic detergent, ionic or zwiterionic detergent, acid and base solutions, hypotonic and hypertonic solutions, alcohols, solvents, enzymes, chelating, physical and miscellaneous agents or any combination of any of the aforementioned solutions and agents. Examples of such detergents or surfactants include Triton X-100, however those of ordinary skill in the art will understand that any suitable decellularization solution may be utilized in the methods described herein.

The incubation, submersion, or flushing of the whole or partial organ in decellularization solution may be performed for durations of, for example and without limitation, 30 minutes to 24 hours, and may be performed at temperatures ranging from 0° C. to 37° C.

Prior to contacting the whole organ or partial organ, for example by flushing the organ with decellularization solution, the whole organ or partial organ is optionally digested with a protease-containing solution, such as a solution comprising an acid protease. As used herein, a protease is an enzyme that breaks down proteins or polypeptides into smaller polypeptides or amino acids. Those of skill in the art are aware of suitable proteases for use in decellularization protocols. However, in non-limiting embodiments, the protease is pepsin or trypsin. In some embodiments, the protease solution is included in the decellularization solution.

In one embodiment, the protease of the protease-containing solution is an acid protease, for example trypsin or pepsin. In a non-limiting example, the organ or partial organ is decellularized by flushing and digestion with a protease-containing solution comprising from 0.005% wt. (percent by weight) to 0.1% trypsin, followed by flushing and treatment with a detergent solution comprising from 0.01% to 5% Triton X-100. In a preferred embodiment, the protease solution comprises 0.02% (by weight) trypsin and the decellularization solution includes 0.1% (by weight) Triton X-100.

In any embodiment, the detergent solution may further comprise a chelating agent, such as 0.001 mM to 10 mM EDTA or EGTA, or, by weight of the decellularization solution, 0.01% to 5% EDTA or EGTA. Prior to decellularization, the whole organ or partial organ may optionally frozen, for example by flash freezing, and thawed, or the organ surface may be cross-linked by exposure to formaldehyde or any other fixative agents.

In an exemplary embodiment, the whole or partial organ is digested with a protease solution for durations ranging from 30 minutes to 24 hours, and digestion occurs at temperatures ranging from 4° C. to 37° C. Following digestion, the digested whole or partial organ is washed, for example by rinsing or flushing, with a wash solution, such as those known to those of skill in the art. Examples of such wash solutions include water, deionized water, cell-free culture medium, phosphate buffered saline (PBS), and combinations thereof. Rinsing/washing may also be performed anytime a step of the decellularization method is completed. Thus, for example, following flushing or other incubation/submersion/immersion in the protease solution, the whole or partial organ may be washed/rinsed with any suitable wash solution and then immersed in or otherwise flushed with the decellularization solution.

According to one embodiment, the organ or partial organ is decellularized by flushing and digestion with a protease-containing solution followed by flushing and treatment with a decellularization solution, followed by disinfecting the ECM construct, again optionally with washing/rinsing steps between the digestion, decellularization, and disinfecting steps. The decellularization also optionally comprises a disinfecting step, e.g., by flushing or otherwise contacting the ECM construct with a solution comprising an appropriate amount of peracetic acid at concentrations ranging from 0.1% to 3% from 10 minutes to 6 hours. In addition, other disinfecting agents may be used, for example and without limitation, antibiotics such as penicillin (1,000-10,000 Units/ml), streptomycin (50-100 μg/m1), gentamycin (1-100 μg/ml) diluted in buffer saline solution (PBS). The whole or partial organ construct cane be exposed to these disinfecting agents for from 30 minutes to 24 hours at temperatures ranging from 4° C. to 25° C. Those of skill in the art will understand that any suitable disinfecting solution or protocol may be used within the spirit of the invention.

One goal for improving host response outcomes is to reduce the formation of thrombi. Accordingly, in an exemplary embodiment, the method of preparing a whole organ or partial organ ECM construct comprises digestion and decellularizing the whole or partial organ as described above, and providing, for example by submersion, immersion, incubation, or flushing the vasculature with, an anticoagulant such as a protein-associating composition (e.g. N-hydroxysucinnimide (NHS)-heparin), such as a polymer-based composition (e.g. NHS-poly(ethylene glycol) (PEG) or equivalent compositions).

As used herein, the term “polymer” includes copolymers, block copolymers, homopolymers, and modified polymers. The composition comprises a non-reactive moiety (e.g. PEG) attached to an amine- or ECM-reactive group (e.g. NHS). A polymer is prepared by polymerization of one or more monomers by any useful polymerization method, such as radical polymerization, such as controlled-radical polymerization, living polymerization, e.g., atom-transfer radical polymerization, though poly(C₁₋₆ alkylene oxide) polymer are typically produced by ionic mechanisms—both cationic or anionic mechanisms—such as in the case of polymerization of ethylene oxide in water. A “residue” is an incorporated monomer. By “attached”, unless indicated otherwise, it is meant linked or covalently bonded. The non-reactive moiety is biocompatible—that is, it does not substantially inhibit cell growth and differentiation and implementation of the methods of producing a whole or partial organ ECM construct as described herein. By “non-reactive”, it is meant that a moiety essentially does not covalently bind, react or link to the whole or partial organ ECM construct under physiological conditions, such as in water, cell culture medium, blood, serum, plasma, PBS, and/or saline.

Non-limiting examples of a non-reactive moiety include polyethers, such as a polyoxyalkylene polymer, such as poly(C₁₋₆ alkylene oxide) polymers or copolymers where two or more different C₁₋₆ alkylene oxide monomer residues are incorporated into the poly(C₁₋₆ alkylene oxide) polymer. “Alkylene” refers to a saturated bivalent, linear or branched, aliphatic hydrocarbon radical, such as methylene (—CH₂—), ethylene (e.g., —CH₂—CH₂—), propylene (e.g., —CH₂—CH₂—CH₂—), tetramethylene (e.g., —CH₂—CH₂—CH₂—CH₂— or —CH₂—CH₂—CH₂(CH₃)—), etc. An exemplary polyether or poly(C₁₋₆ alkylene oxide) polymer is polyoxyethylene (PEG), having the structure —(O—CH₂—CH₂)ₙ—OH, in which n is an integer greater than or equal to 2. In one non-limiting embodiment, n is 2-50. Other examples of the poly(C₁₋₆ alkylene oxide) polymer moiety include polypropylene glycol (PPG; H—(O—CH₂—CH₂—CH₂—CH₂—)ₙ—OH) or polytetramethylene glycol (PTMEG; H—(O—CH₂—CH₂—CH₂(CH₃)—)ₙ—OH), in which n is an integer greater than or equal to 2. In one non-limiting embodiment, n is 2-50. In addition, polyether-containing block polymers, comprising blocks of different polyether, polyoxyalkylene or poly(C₁₋₆ alkylene oxide) blocks, such as PEG-PPG-PEG block copolymers may be used as the non-reactive moiety. The non-reactive moiety, such as a polyether, is modified with an amine-(or ECM) reactive moiety, such as NHS, isocyanate (NCO), carboxyl (COOH), aldehyde (C═O), or chloride (Cl) groups. Suitable block copolymers can be formed using living radical polymerization techniques as well as click chemistry techniques, as are known to those of skill in the art.

Other compositions suitable for use in the protein-associating composition include, without limitation: zwitterionic moieties (e.g., phosphorylcholine (PC), sulfobetaine (SB), carboxybetaine (CB)), macromolecules or polymers with amine reactive groups (N-hydroxysucinnimide (NHS), isocyanate (NCO), carboxyl (COOH)) (e.g., PC-NHS, PC-NCO, SB-PEG-NHS, PC-COOH, SB-COOH, PEG-PPG-PEG-NHS, PEG-SB-NHS compositions), or poly[N-p-vinylbenzyl-4-O-β-D-galactopyranosyl-D-gluconamide]-co-valine (PVLA-co-VAL), or PVLA-co-VAL-PEG-NHS to inhibit acute thrombosis in damaged vascular and biomaterial surfaces. These compositions can be additionally bound to biotin, for example for detection. An exemplary polymer including biotin is poly(ethylene glycol) (N-hydroxysuccinimide 5-pentanoate) ether 2-(biotinylamino)ethane.

In addition to the above-identified compounds that may be used to reduce and/or eliminate formation of thrombi in the whole or partial organ ECM construct, additional suitable compounds may be found in U.S. Pat. No. 5,977,252, including, for example and without limitation, compounds including ester, anhydride (including N-carboxy anhydride), isocyanate (as described above), aldehyde, tosylate, tresylate or epoxide groups/moieties. Reactive end groups that will not release small molecules or toxic molecules upon the covalent attachment of the polymer are preferred. To this end, cyclo-esters, cyclo-anhydrides and isocyanates are also suitable reactive groups to attach to the end of the polymer and effectuate the covalent modification.

In another embodiment, the protein associating composition comprises an NHS moiety covalently linked to a non-reactive, biocompatible polymer moiety. In yet another embodiment, the NHS moiety is linked (covalently bonded) to a PEG moiety. In an exemplary embodiment, the protein-associating polymer comprises N-hydroxysuccinimide (NHS)-modified poly(ethylene glycol) (PEG).

The compositions described above for use as anticoagulants in decellularized organs produce more than 80% of adequate re-endothelization of all vessels in the whole or partial organ, and re-epithelialization up to 30-40% of the whole or partial organ. These levels are possible because the compositions reduce the formation of thrombi.

The step of immersion/submersion/incubation/flushing of the whole organ or partial organ with the above-described protein-associating compositions may be conducted prior to ex vivo population of the whole organ or partial organ with cells, or after ex vivo population of the organ or partial organ with cells, or, for example, immediately before implantation of the cell-populated organ into a patient. Exposure of the whole or partial organ to the polymer may be for durations ranging from 30 minutes to 24 hours, and may occur at temperatures ranging from 4° C. to 37° C. and can be done under flow conditions ranging from 1 ml/min to 100 ml/min, or in static conditions. Those of skill in the art will appreciate that reaction times will vary based on the protein-associating polymer that is used.

Also provided herein is an extracellular matrix (ECM) organ structure, comprising a decellularized whole organ or partial organ substantially comprising native three-dimensional ECM structure, and an anticoagulant, such as a protein-associating composition, such those described above, dispersed within and/or coating the native ECM structure (e.g., comprising essentially all macro-structural elements of the organ or partial organ from which the organ structure is prepared). The organ structure optionally comprises cells. For example, one embodiment is a commercial product comprising a decellularized organ structure comprising the anticoagulant. In another embodiment, the commercial product is the organ structure populated with cells, such as a patient's autologous cells for transplantation into the patient, and comprising the anticoagulant, which is applied to the organ structure after population of the organ structure with cells and prior to implantation thus coating exposed ECM material in the organ structure.

In one embodiment, the ECM organ structure is prepared according to any method described herein, or any suitable method known to those of skill in the art to provide a whole or partial organ ECM construct with low immunogenicity and suitable for implantation, and provided with (for example coated with) an anticoagulant polymer as described herein.

Also provided is a method of producing an artificial organ, comprising, prior to or after administration of the anticoagulant, perfusing the ECM whole or partial organ structure, as described herein, with one or more cells, such as, for example, primary cells (e.g., hepatocytes), multipotent cells and/or pluripotent cells, for example progenitor cells or stem cells, as are broadly known in the field. The cells may be, according to certain embodiments, orthotopic, autologous, allogeneic and/or xenogeneic. The artificial organ (organ structure) is implanted in a patient in need thereof, for example and without limitation, a liver ECM structure as described herein is perfused with hepatocytes and incubated, for example as described below, flushing the organ structure with the anticoagulant prior to, for example immediately prior to, implantation of the organ structure in a patient.

### EXAMPLES

Quality Assessment Protocols to Evaluate Whole Organ Liver Decellularization

Protocols for whole liver decellularization. Different detergents (SDS, trypsin and Triton X-100) were evaluated for their effect on the organ ECM. System criteria for evaluation were based on the preservation of structural and extracellular proteins, DNA remnants, the presence of growth factors, and integrity of the collagenous capsule covering the external surface of the liver (i.e. Glisson's capsule). For instance; to quantitate the desired features of an extracellular matrix after liver decellularization using multiphoton imaging 5 75-90 μm (depth) beyond the liver surface (Glisson's capsule), it was found that 0.02% trypsin and 0.1% Triton X-100 maintained the structure, orientation and density of the collagen better than use of 0.02% trypsin and 3% Triton X-100 as decellularization. In normal livers, collagen fibers were long and widely separated by the cellular content. Moreover, scanning electron microscopy (SEM) images confirmed the presence and higher collagen density preserved when 0.02% trypsin and 0.1% Triton X-100 protocol was used. A meticulous analysis of the surface of the Glisson's capsule showed complete integrity of the Glisson's capsule in both decellularization protocols. It was found that 100% of the fibrillar collagen of native liver was retained after 0.02% trypsin and 0.1% Triton X-100 decellularization. It was also found that residual DNA content in both decellularization protocols was less than 10%. Based on these results, 0.02% trypsin/0.1% triton was selected as being most optimized and, as expected, fibronectin and laminin components of the basement membrane were preserved. The criteria that define a successful organ decellularization process are poorly understood. From empirical experience it is known that the goal is to maintain the quality and quantity of the collagen content and the preservation of the basement membrane components as close to normal liver as possible. Under these circumstances, an organ scaffold can be produced with no leakage in flow culture conditions.

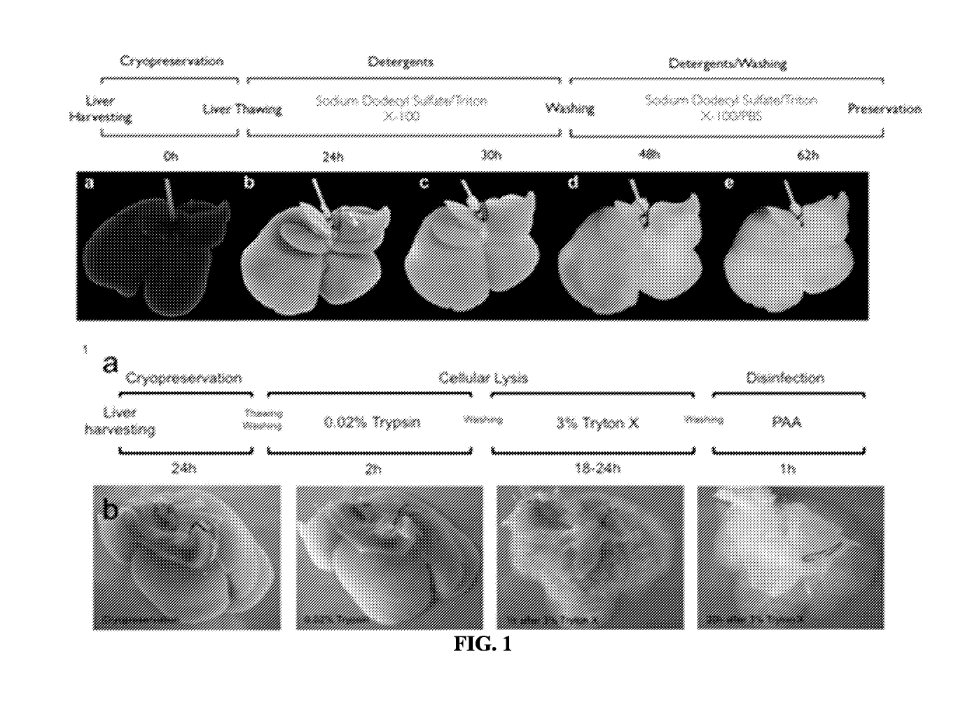

FIG. 1 shows variations of useful whole organ decellularization protocols suitable for use in the methods of the present invention. As described previously, other decellularization protocols are known (WO 2012/031162; WO 2011/002926; EP 2501794; U.S. Pat. No. 8,470,520). However, as described below, a decellularization protocol described herein provides optimal results.

A new methodology to easily check the quality of the decellularization process based on the previous criteria was developed. The results show that an optimized decellarization protocol consists of a combination of 0.02% trypsin and 0.1% Triton X-100/0.05% EGTA.

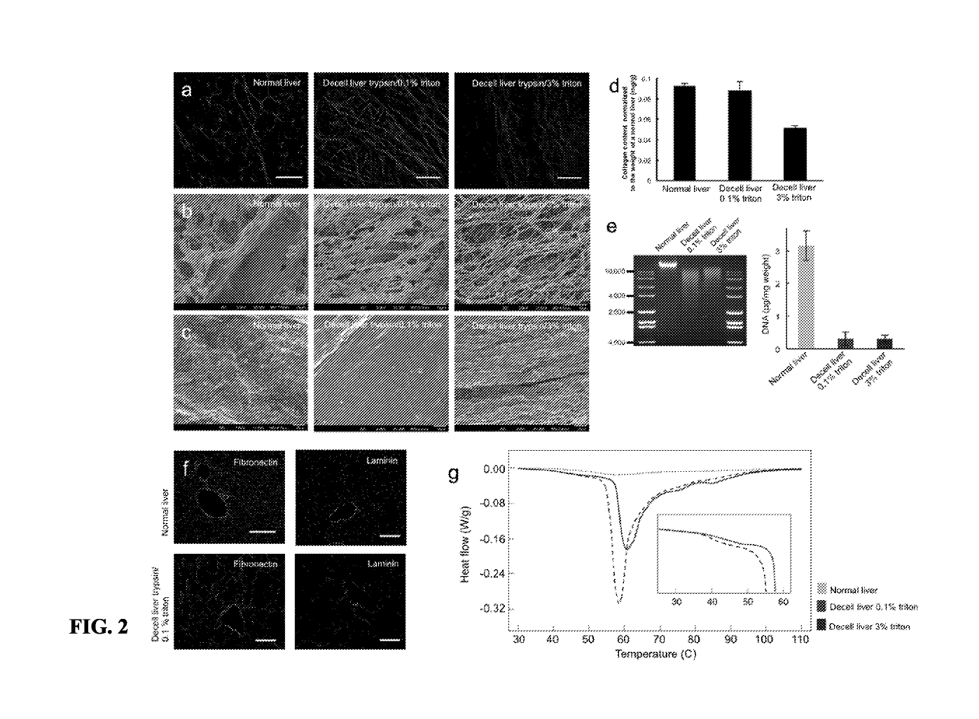

FIG. 2 shows results of this optimization and characterization of decellularized rat livers. Panel (a) shows representative images of multiphoton microscopy and of normal and decellularized rat livers observed in at least three specimens. Panel (b) shows SEM images of extracellular matrix within the parenchyma. Panel (c) shows Glisson's capsule of normal and after liver decellularization. At least 5 different liver specimens and 7 liver lobes per each were analyzed per group. Panel (d) shows collagen content of normal and decellularized rat liver using 3% and 0.1% triton X-100 solutions (n=3), error bars, mean±s.d. Panel (e) shows DNA content of normal and decellularized rat liver using 3% and 0.1% triton X-100 solutions (0.1% vs 3%, p=0.999 by one-way ANOVA, Tukey-Kramer) (n=3), error bars, mean±s.d. Panel (f) shows a comparison of normal liver (top) and decellularized rat liver (bottom). Left to right: fibronectin (red) and laminin (red) staining. Sections were counterstained with DAPI (blue). Panel (g) shows thermograms of normal liver (green) and decellularized liver using 3% (blue) and 0.1% (red) triton X-100 solutions. Samples were scanned at 3° C./min between 2° C. and 125° C. Plotted lines were the weight-averaged curves of four samples in each group. The inset shows the lower temperature shoulders of extracellular tissue matrices. The total denaturation enthalpy when using low concentrations of triton is 45.6±5.0 J/g (n=4). The total denaturation enthalpy for extracellular matrices derived with 3.0% Triton X-100 is 55.8±4.7 J/g (n=4), a value that is significantly higher than that of the extracellular tissue matrix derived with 0.1% Triton X-100 (p=0.026) and is similar to purified collagen. Scale bars: 10 μm (a) and 50 μm (f).

The liver decellularization protocol described herein preserves the structure and alignment of the collagen fibers as shown and analyzed by multiphoton fluorescence microscopy, this technique allows imaging several hundreds of micrometers deep into biological samples as scattering of red-shifted light for collagen fibers (FIG. 2, panel a) and thus, the collagen fiber layer thickness, density, and orientation can accurately be determined as a function of radial position and quadrant location. FIG. 2, panel a shows representative images of multiphoton microscopy and of normal and decellularized rat livers observed in at least three specimens. Imaging acquisition began at the margin of the Glisson capsule on the coverslip and extended to depths of 75-90 μm behind the Glisson capsule. Birefringence at 350-450 nm reveals the morphology and arrangement of collagen fibers (red). As shown in FIG. 2, panel a, reduction in the concentration of Triton X-100 from 3% to 0.1%, significantly improved the preservation of collagen fiber layer thickness, density, and orientation. Moreover, DNA content was analyzed and gel electrophoresis confirmed that the DNA content of the decellularized liver matrix was degraded and reduced 10-fold when compare to the DNA total content of a normal liver (FIG. 2, panel e). As indicated previously, elimination of substantially all or all nucleic acids will improve outcomes in the host response. For example, and without limitation, the amount of DNA remaining after decellularization should be equal to or less than 50 ng/mg of tissue. With regard to residual DNA, the protocol of 0.02% trypsin and 0.1% Triton X-100 showed residual nucleic acids of less than 10% (FIG. 2, panel e).

The method and condition with which the liver tissue is decellularized will have profound impact on both the structure and biological composition of extracellular tissue matrices. Differential scanning calorimeter (DSC) analysis is a useful tool for assessing the extent of decellularization and effects on structure/composition. FIG. 2, panel g shows thermograms of extracellular tissue matrices yielded with 0.1% or 3.0% Triton X-100 solutions. Fresh liver tissue is primarily composed of cellular elements, of which the enthalpy of thermal transitions is very small relative to extracellular matrix components as indicated by a more or less flat thermogram. Significant differences are observed between two extracellular tissue matrices derived with 0.1% and 3.0% Triton X-100. The thermogram of extracellular matrix derived with 0.1% Triton X-100 shows at least four transitional events during a calorimetric scan between 2° C. and 125° C., with the onset denaturation temperature at ˜40° C., 58° C., 70° C. and 80° C., respectively (FIG. 2, panel g). The total denaturation enthalpy is 45.6±5.0 J/g (N=4). The transitional events at 70° C. and 80° C. are absent in the extracellular matrix derived with the high concentration of Triton X-100, and the thermogram is also shifted to lower temperature, indicating a less stable extracellular tissue matrix (57.5±0.3° C. vs 54.9±0.9° C., N=4, p=0.002). The total denaturation enthalpy for extracellular matrices derived with 3.0% Triton X-100 is 55.8±4.7 J/g (N=4), a value that is significantly higher than that of the extracellular tissue matrix derived with 0.1% Triton X-100 (p=0.026) and is similar to purified collagen. The DSC data suggest that 3.0% Triton X-100 removes more thoroughly many inherent extracellular elements, and deeply affects its biological compositions. Fresh liver tissue is primarily composed of cellular elements, where the enthalpy of thermal transitions is very small relative to that of the extracellular matrix components, where it is indicated by a relatively flat thermogram.

As the results in FIG. 2 show, the use of 0.02% trypsin and 0.1% Triton X-100 maintains superior collagen structure, orientation, and density in the decellularized whole/partial organ. In addition, collagen density was highest when that protocol was utilized (FIG. 2, panel b), and the integrity of Glisson's capsule was maintained in its entirety (FIG. 2, panel c). Lastly, 100% of the fibrillary collagen of native liver was retained using this optimized decellularization protocol (FIG. 2, panel d). The preservation of the basement membrane is an important cue for the new formation of endothelium. The decellularized livers expressed the ECM proteins fibronectin and laminin, indicating that both structural and basement membrane components were retained at a similar organization found in native liver (FIG. 2, panel c) and were exceptionally preserved (FIG. 2, panel 1).

Liver Graft Re-Cellularization and Culture-Perfusion System

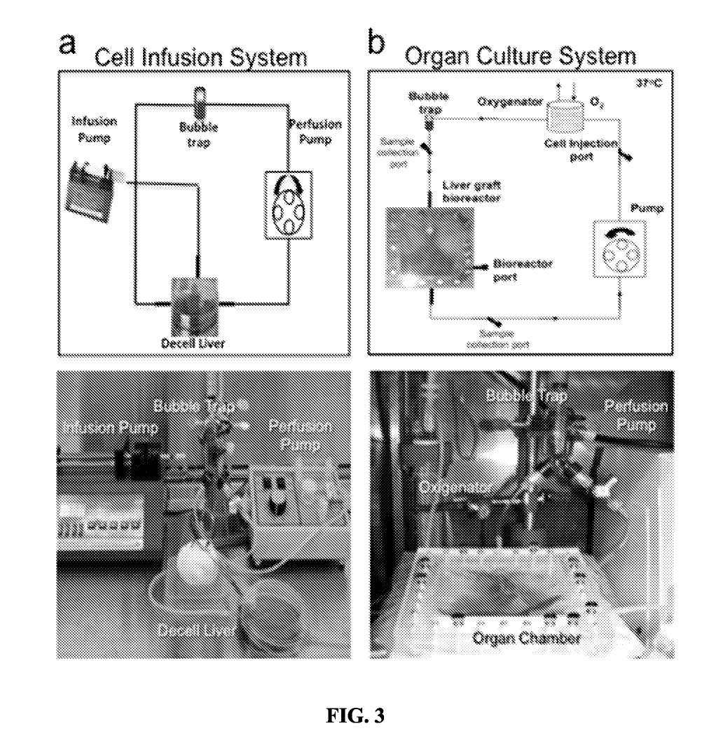

Two different systems useful for re-cellularization are shown in FIG. 3: a cell infusion system for controlled re-cellularization through the portal vein, bile duct, and/or vena cava (FIG. 3, panel a) and an organ culture system for continuous long-term graft culture (FIG. 3, panel b). In either system the medium is changed daily. A total volume of 50 ml medium is recirculated in the perfusion system. The presence of a functional vascular bed in the decellularized liver matrix offers the ability to control hepatocyte engraftment and characterize liver-specific metabolic function in vitro. Freshly isolated primary rat hepatocytes are introduced via portal vein perfusion recirculation using a 4-step protocol. Re-cellularized rat livers with only primary rat hepatocytes can be perfused for up to 2 weeks at 37° C. and exhibit suitable cell viability and function over time (Uygun et al. “Organ reengineering through development of a transplantable re-cellularized liver graft using decellularized liver matrix.” (2010) Nat Med. 16(7):814-20).

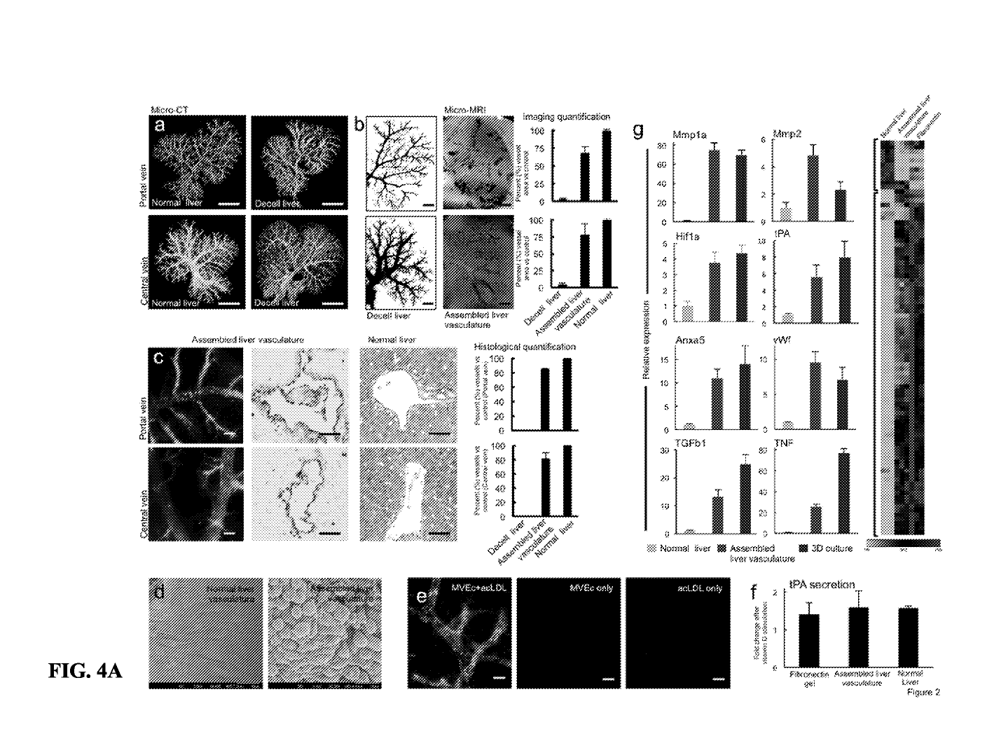

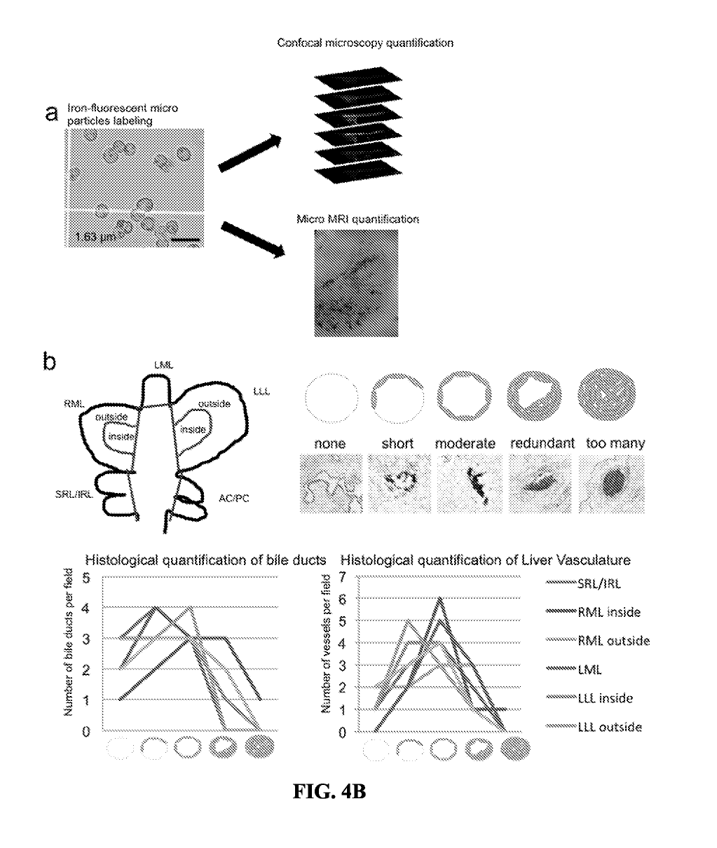

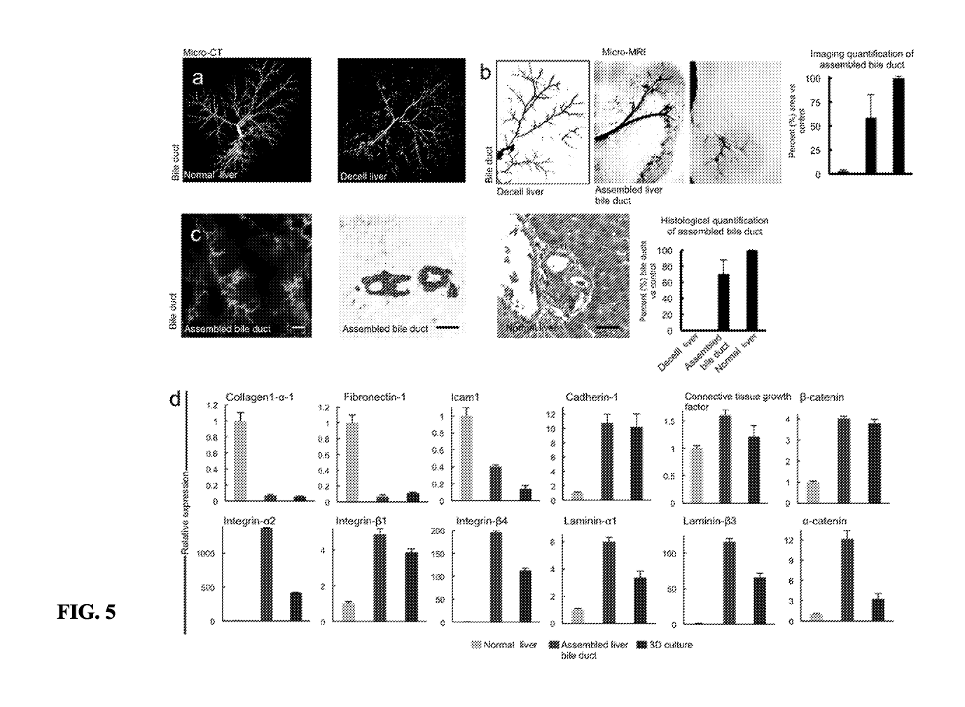

Optimization and evaluation system of vascular re-endothelialization and bile duct re-epithelialization. Ultimately reconstruction of liver grafts in vitro also requires the addition of liver non-parenchymal cells. Previous work has demonstrated intact vasculature using corrosion cast technique. FIGS. 4 and 5 show the results of vascular re-endothelialization.

FIG. 4A, panel a shows 3D micro-CT angiography of normal and decellularized livers vascular compartments (portal and central vein). Panel (b) shows representative micro-MRI images of micron-sized iron oxide particle-labeled endothelial cells seeded into the portal and central vein of decellularized livers. Quantification of the liver vasculature repopulation is also shown compared to control paired micro-CT images. For pairing images, planar (2D) images (approximate slice thickness 1 mm) were obtained for each liver lobe. To allow accurate definition in image pairing the major branch of the portal vein, central vein or bile duct was selected for each lobe and manually traced and divided into interbranch segments for anatomical image pairing. Segments were traced beginning at the largest portal vein, central vein or bile duct and moving along the major branch to the smallest. The quantitative analysis of the obtained images of the structures of the liver lobes was performed using the OsiriX image processing software program. Panel (c) shows representative fluorescent confocal microscopy images of the same micron-sized iron oxide particles-labeled endothelial cells assembled portal and central veins of decellularized livers and the corresponding images of histological sections stained with hematoxylin and eosin. Histological quantification of assembled whole organ vasculature is also shown. Panel (d) shows SEM images of normal liver vasculature and assembled liver vasculature. At least 3 different liver specimens and 3 liver lobes per each were analyzed per group. Panel (e) shows representative fluorescence images of assembled liver vasculature (portal and central vein) using micro-vascular endothelial cells (MVEc) exposed to AlexaFluor 488-labeled ac-LDL and images of control experiments (MVEc only and AlexaFluor 488-labeled ac-LDL only in acellular decellularized liver). Panel (f) shows Vitamin D-stimulated secretion of tissue plasminogen activator (tPA) of microvascular endothelial cells cultured on fibronectin gel, assembled liver vasculature and normal liver (p=0.5788, Student's t-test) (n=3) error bars, mean±s.d. (g) Gene expression analysis of assembled liver vasculature (portal and central vein) with only microvascular endothelial cells compared to normal liver and MVEc fibronectin gel culture for endothelial cell biology markers (n=3 each). Normalized gene expression of matrix metallopeptidase 1a and 2 (Mmp1a, Mmp2), Hypoxia-inducible factor 1-alpha (Hif1a), tissue plasminogen activator (tPA), Annexin A5 (Anxa5), Von Willebrand factor (vWF), Transforming growth factor beta-1 (TGFb1), Tumor necrosis factor (TNF). All error bars represent+s.e.m. (n=3). Scale bars: (a) 1 cm, (b) 4 mm, (c) from left to right 100 μm and 50 μm for hematoxylin and eosin photographs, (d) 100 μm.

FIG. 4B, panel a shows a schematic representation of two different types of anatomical remodeling after repopulation of the vascular and bile duct systems using micron-sized iron oxide particle-labeled endothelial cells and cholangiocytes. An example for quantitative analysis is shown for the biliary tree assembled in the decellualrized rat liver. The rat liver lobes were divided and images of each lobe were obtained by either confocal microscopy or micro-MRI, major branches of the biliary tree were selected, manually traced, and at least 5 different depths images were analyzed at each branch point. The surface area of each bile duct segment was compared to paired images at the same depth and positioning of three-dimensional microCT images of the intrahepatic biliary of normal rat livers that were produced by injecting contrast agent for biliary tree visualization into the common bile duct as described in detail in Methods. Panel (b) shows a schematic representation of the histological quantification of repopulation of bile ducts and vasculature (portal or central vein). The entire repopulated rat liver was divided into different sections for evaluation purposes; superior right lobe (SRL), inferior right lobe (IRL), right medial lobe “outside” or “inside” (RML), left medial lobe (LML), left lateral lobe “outside” or “inside” (LLL), anterior caudate lobe (AC) and posterior caudate lobe (PC). H&E sections of each lobe were traced manually and assigned a level of coverage (none, short, moderate, redundant, too many) and quantified per field. An example of for quantitative histological analysis is shown.

FIG. 5, panel a shows 3D micro-CT angiography of normal and decellularized liver bile duct. Scale bar, 4 mm. Panel (b) shows representative micro-MRI images of micron-sized iron oxide particle-labeled cholangiocytes seeded into the bile duct of decellularized livers at different depth levels. Quantification of the liver bile duct repopulation is also shown compared to control paired micro-CT image. Panel (c) shows representative fluorescent confocal microscopy images of the same micron-sized iron oxide particle-labeled cholangiocytes assembled bile duct of decellularized livers and the corresponding images of histological sections stained with hematoxylin and eosin. Histological quantification of assembled whole organ bile ducts is also shown. Panel (d) shows normalized gene expression of cell-cell and cell-matrix adhesion molecules (n=3). Scale bars: (c) from left to right 100 μm and 50 μm for hematoxylin and eosin (H&E) photographs.

As described above, micro computed tomography (CT) was also utilized to characterize the architectural vasculature of the bile duct, portal vein and central vein (FIG. 4A, panels a, b; FIG. 5, panels a, b) of decellularized livers compared to fresh livers. 40 million iron-fluorescent-microparticle-labeled microvascular endothelial cells via portal and central vein structures by perfusion through a recirculation system. All the specimens were scanned by micro CT scanner to provide the three-dimensional images of the intrahepatic biliary tree, portal vein and central vein vascular systems. In order to evaluate and optimize different cell seeding protocols of vascular and bile duct re-cellularization, the rat lives was divided anatomically and functionally into 7 different segments for histological evaluation of the re-cellularization protocols. At least 10 different fields are evaluated histologically (H&E stain) per each liver segment to test different protocols for vascular re-endothelialization or bile duct re-epithelialization. This system allows serial evaluations of the re-cellularization protocols for further optimizations. Human liver non-parenchymal cell lines (human sinusoidal endothelial cell and human bile duct cell line) and also rat primary liver cells were used to optimize re-cellularization protocols. It was found that up to 80-90% of the vessels in the whole liver were adequately re-cellularized with endothelial cells (FIG. 4A, panels a-c) and about 60-80% of the bile ducts were adequately re-cellularized with cholangiocytes using the best-optimized protocols (FIG. 5, panels a-c). This data demonstrate that hepatocytes, endothelial and bile duct cells can be seeded in the re-cellularized grafts with great efficiency and limited damage.

Assembly of Whole-Organ Liver Vasculatures and Bile Duct

In order to corroborate the histological quantification of the whole organ re-cellularization protocols in a more systematic fashion, micro Magnetic Resonance Imaging (MRI) was used. The liver was divided in different segments (SRL/IRL; superior right lobe/inferior right lobe, RML; right median lobe, LML; left median lobe, LLL; left lateral lobe, AC/PC; anterior caudate lobe/posterior caudate lobe) and obtained 2D images of the intrahepatic biliary tree, portal and central vein vasculature of each segment (FIG. 4A, panel a). Decellularized livers were re-cellularized using bile duct epithelial cells or endothelial cells that were previously labeled with fluorescent iron nano-particles. At least 10 different 2D images were obtained for each segment. The quantitative analysis of the two-dimensional images of the intrahepatic biliary tree was performed using Image J software. Volume rendering and maximum intensity projection was displayed at various angles of view and threshold voxel values. Average voxel size was 100 to 500 μm and images of up to 50 slices were rendered for each specimen. The measurement of cross-sectional area of the bile duct, portal vein and central vein segments was made by use of brightness area product. The length of bile duct, portal vein and central vein segment was measured as a straight-line distance between bifurcations. The volume of each bile duct segment was calculated from volume=length×cross-sectional area. The surface area of each bile duct, portal vein or central vein segment was given by surface area=2×length×root square (cross-sectional area×π).

The best perfusion protocol results in repopulation of 68±9% of the portal vein and 78.3±16% of the central vein, and microscopy analysis also confirmed that microvascular endothelial cells lined the interior of the portal and central veins. Histological quantification revealed that 86±3% of the portal vein system and 81±9% of the central venous system were repopulated (FIG. 4A, panel c; FIG. 4B). Ultrastructural analysis of the assembled vasculature confirmed that microvascular endothelial cells lines the inside of the blood vessels with cells flat and thin with abundant microvilli, all endothelial cells were attached and some partially flattened (FIG. 4A, panel d).

The resulting evaluation was compatible with the histological evaluation previously developed. This novel, powerful imaging technique is capable of providing a systematic three-dimensional or two-dimensional quantitative analysis of the normal intrahepatic biliary tree, portal and central vein as well as the corresponding evaluation of the re-cellularization of whole liver scaffolds (FIG. 4A, panels a-c; FIG. 5, panels a-c).

Functional evaluation of liver vascular re-endothelialization. Most importantly, to date the major challenge in tissue/organ engineering (including liver) has so far been limited graft survival after transplantation. That is, the main gap that prevents advancement of the field is the lack of strategies to prevent acute thrombosis after graft transplantation. Thus, the development of a functional liver vasculature is imperative to achieve long-term survival of engineered organs. It has previously been demonstrated that intact vasculature using corrosion cast technique. Additionally, micro computed tomography has been performed to characterize the architectural vasculature of the decellularized liver. In addition, systems to monitor liver re-cellularization of the entire vasculature and the bile duct based on imaging techniques (micro computed tomography and magnetic resonance imaging) have been developed. Based on these techniques. It was found that up to 80-90% of the vessels in the all liver were adequately re-cellularized with endothelial cells and about 60-80% of the bile ducts were adequately re-cellularized with cholangiocytes using the methods described herein. This data demonstrate that hepatocytes, endothelial and bile duct cells can be seeded in the re-cellularized grafts with great efficiency and limited damage. The functionality of the engineered liver vasculature in the organ culture system was analyzed by the evaluation of the intake of acetylated low-density lipoprotein (ac-LDL) using confocal microscopy, a characteristic of endothelial cells to use the “scavenger cell pathway” of LDL metabolism. Additionally, tissue plasminogen activator (tPA) secretory ability was measured in the culture medium (a protein involved in the breakdown of blood clots) after the exposure of vitamin D, and endothelial gene expression was also characterized.

Acetylated low-density lipoprotein (ac-LDL) is known to be incorporated into microvascular endothelial cells. Uptake of fluorescence-labeled ac-LDL was evaluated and, as expected, the newly engineered liver vasculature took up Dil-labeled (Dil is available commercially, for example from Life Technologies) acetylated low-density lipoprotein (Ac-LDL), a specific function of endothelial cells in vitro, and demonstrated the detailed three-dimensional structure of the portal and central venous system (FIG. 4A, panel e). Additionally the ability of the engineered liver to secrete the acute-reactant tissue plasminogen activator after stimulation with vitamin D (1×10⁻¹² M) was examined The newly engineered liver vasculature reactive secretion of tPA was about a 1.5 fold increase (FIG. 4A, panel f). Microvascular endothelial cells cultured in a static 3D configuration using fibronectin had similar response. These results demonstrate that the microvascular endothelial cells were functional in the engineered liver.

Analysis of the expression of endothelial cell-related genes via quantitative RT-PCR after whole liver vasculature engineering revealed that expression levels of genes related to angiogenesis (endothelial cell growth and remodeling) and coagulation in the re-cellularized liver vasculature were similar to those measured in 3D-fibronectin cultures (FIG. 4A, panel g). As expected in this culture conditions (only microvascular endothelial cells) gene transcription levels in were overall higher than those of liver. However, inflammatory genes transforming growth factor beta 1 (TGFb1) and tumor necrosis factor (TNF) were expressed in the re-cellularized liver at lower levels compared to those in 3D-fibronectin cultures.

Next, it was determined whether the biliary system could be re-assembled in the decellularized livers, a prerequisite for producing a functional liver graft. The matrix of the biliary system was repopulated with a total of 6×10⁶ bile duct epithelial cells through the matrix of the main bile duct. For quantification, iron-fluorescent-microparticle-labeled cells were used. Optimization was based on the percentage of the bile duct area lined by infused cells. Micro-imaging revealed that 59±24% of the bile ducts could be repopulated (FIG. 5, panels a, b). Confocal microscopy revealed a branched, tree-like bile canaliculi network throughout the liver (FIG. 5, panel c), and quantitative histological scoring showed that 70±18% of the bile duct system was repopulated (FIG. 5, panel c). Expression of cell adhesion molecules showed that the biliary system generally resembled cultured bile duct epithelial cells in a static 3D configuration, although some cell-ECM interaction genes were highly expressed only in the assembled liver bile duct configuration (FIG. 5, panel d) as the scaffold structure is likely to provide the necessary architecture for rearrangement, incorporating the architecture of the bile duct and the natural bile duct ECM composition facilitating interactions.

Assembly and Function of Bioengineered Liver

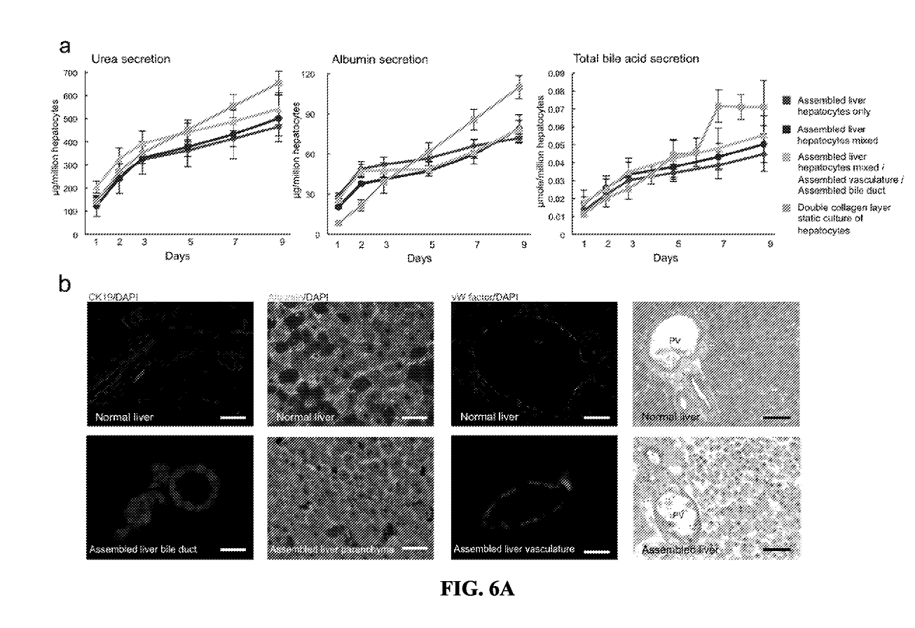

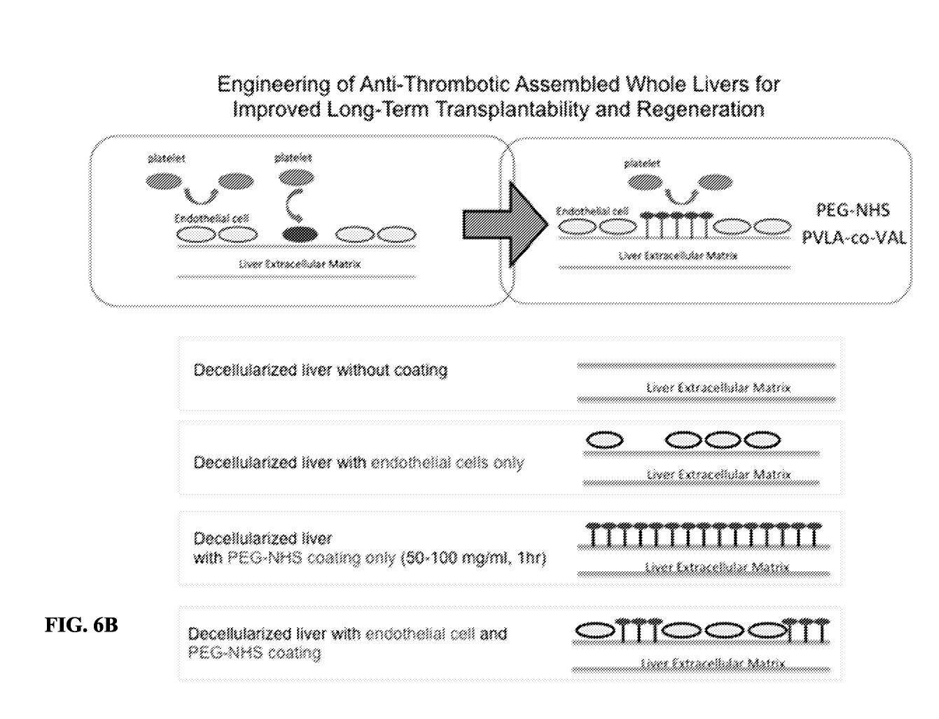

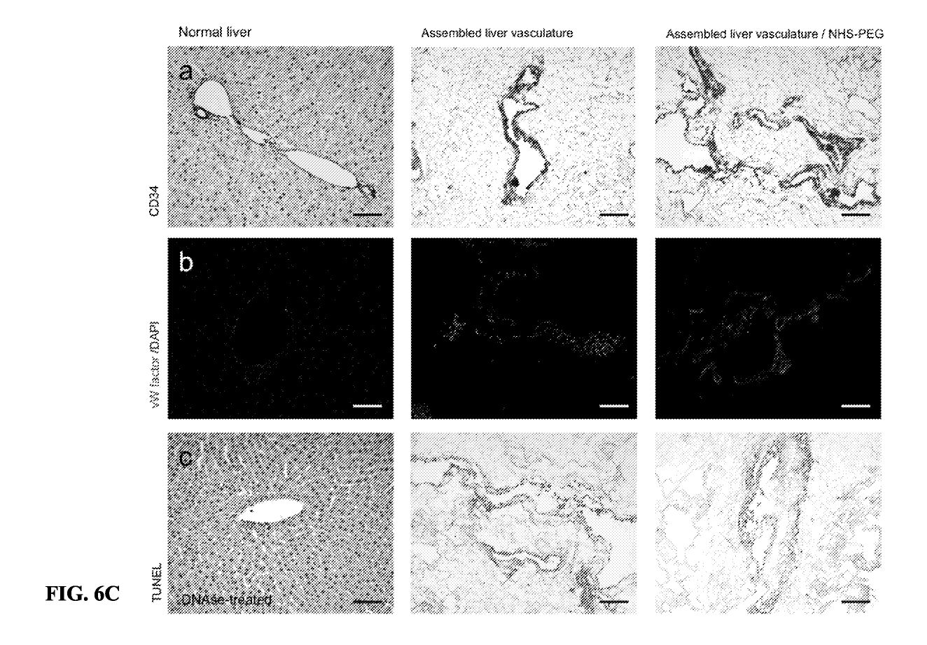

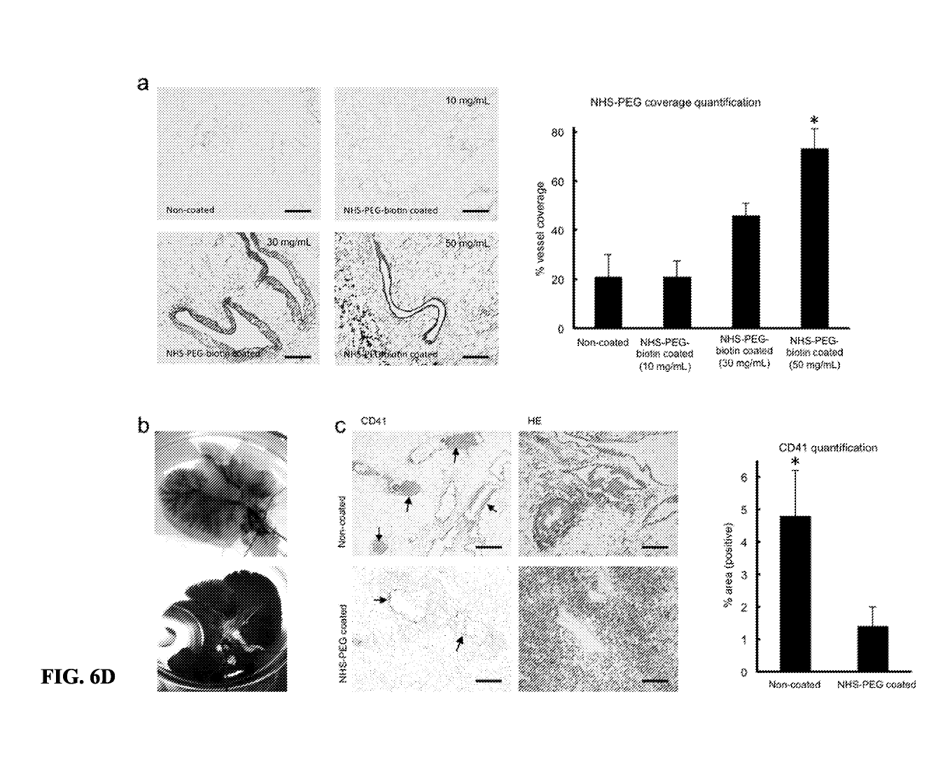

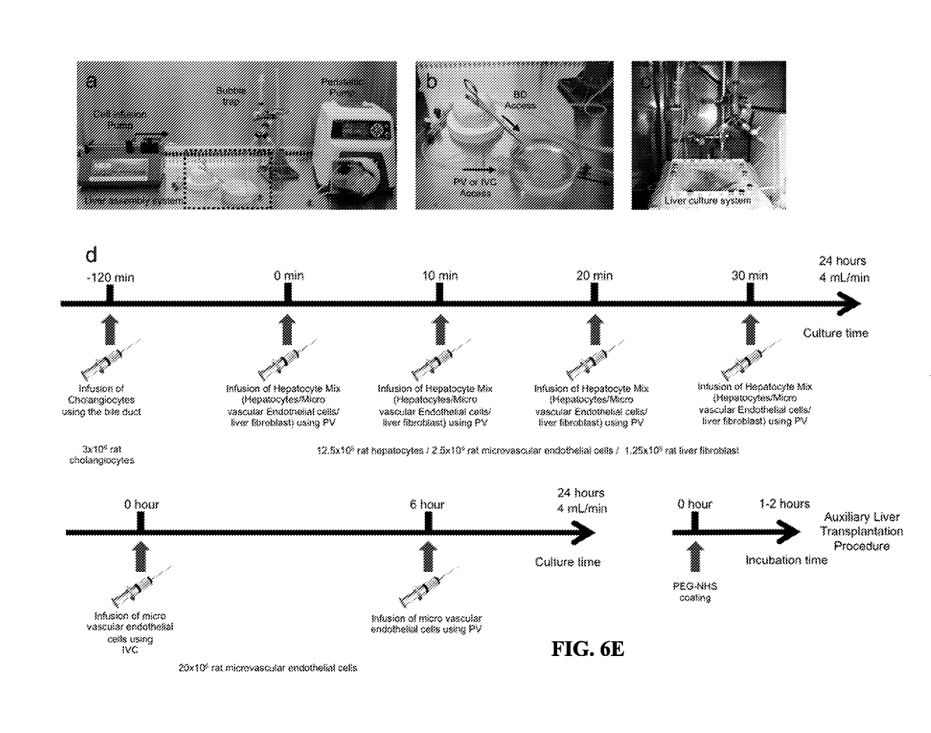

To test hepatic function, the functional characteristics of the three engrafted cellular compartments (hepatocytes, bile duct cells, and microvascular endothelial cells) in the decellularized matrix were also analyzed. FIG. 6 shows results of these functional tests.

FIG. 6A, panel a shows urea secretion (p<0.05, for assembled liver hepatocytes mixed/assembled vasculature/assembled bile duct group versus assembled liver hepatocytes only, one-way ANOVA, Bonferroni), albumin synthesis (*p<0.05, double collagen layer static culture of hepatocytes vs other groups, one-way ANOVA, Bonferroni) and total bile acid secretion of assembled liver using combined repopulation protocols (n=4). Panel (b) shows immunohistochemical staining of the assembled liver compartments (bottom) in comparison to normal liver (top); left to right:, Cytokeratin 19 (CK19) (red), albumin (green) and Von Willebrand (vW) factor (red) and H&E. Sections were counterstained with Hoechst 33258 (blue). Scale bars: 50 μm (b).

FIG. 6C, panel a shows decellularized liver matrix treated with different doses of NHS-PEG-biotin and histological quantification of vessels covered with NHS-PEG-biotin (*p<0.0001 by one-way ANOVA, Turkey-Kramer). Panel (b) shows representative photographs of NHS-PEG treated decellularized livers and directly perfused with portal blood flow. Panel (c) shows immunohistochemical staining for CD41 (platelet marker) and H&E staining of control and NHS-PEG treated decellularized liver matrix after perfusion of portal blood flow. Quantification of CD41 positive areas is also shown (p<0.0001 by Student's t-test). All error bars represent s.e.m. Scale bars (a,c) 100 μm.

FIG. 6D, panel a shows decellularized liver matrix treated with different doses of NHS-PEG-biotin and histological quantification of vessels covered with NHS-PEG-biotin; Panel (b) shows representative photographs of NHS-PEG treated decellularized livers and directly perfused with portal blood flow; and panel (c) shows immunohistochemical staining for CD41 (platelet marker) and H&E staining of control and NHS-PEG treated decellularized liver matrix after perfusion of portal blood flow.

FIG. 6E, panel a shows liver assembly system for in vitro repopulation of decellularized liver grafts; Panel (b) shows perfusion chamber with cannulas to access portal vein (PV), inferior vena cava (IVC) and bile duct (BD) for cell delivery; Panel (c) shows liver culture system assembled of perfusion chamber, peristaltic pump, oxygenator, bubble trap and access ports. Panel (d) shows liver graft assembly protocol.

As described above, hepatic function was analyzed via immunostaining of cytokeratin 19 (CK19) for bile duct cells, albumin for hepatocytes, and Von Willebrand factor for microvascular endothelial cells (FIG. 6A, panel b). The level of immunostaining for these markers in engrafted cells was similar to that in normal livers. The majority of hepatocytes remained near vessel structures with the parenchymal space; microvascular endothelial cells lines the vascular channels and bile duct epithelial cells lined the bile duct channels (FIG. 6A, panel b; FIG. 6C, panel a; FIG. 6D).

To assess the metabolic activity of engrafted hepatocytes, albumin urea synthesis, production, and total bile acid secretion were measured. The cumulative urea, albumin and total bile acids amounts in the re-cellularized liver system were not different within the experimental groups and not higher than hepatocyte sandwich culture during the 9 days culture period (FIG. 6A, panel a). The data shown in FIG. 6A, 6C, and 6D demonstrate that the use of simultaneous assembly of different compartments of the liver (vasculature, bile duct, hepatic parenchyma) does not affect hepatic function.

Long-Term Function and Regeneration Capacity Following Auxiliary Transplantation

Establishment of an auxiliary liver transplantation model in albumin-deficient mutant rats. Previously, the survival of bioengineered decellularized liver grafts has been limited to a few hours as a result of vascular thrombosis or bleeding following the use of systemic anticoagulation. To avoid these complications, liver grafts were bioengineered to incorporate anti-thrombotic activity (FIG. 6B—described more fully below). The ECM-surface was modified with N-hydroxysuccinimide-polyethylene glycol (NHS-PEG) and was conjugated with biotin for detection purposes. 50 mg/mL of NHS-PEG-biotin cover 73±8% of the decellularized liver surface area. To investigate the effects of NHS-PEG on assembled liver vasculature, CD34, Von Willebrand facto, and TUNEL staining were evaluated after 24 h of NHS-PEG treatment. NHS-PEG treatment did not affect the expression of these vascular endothelial cell markers or cell viability (FIG. 6C, panels a-c).

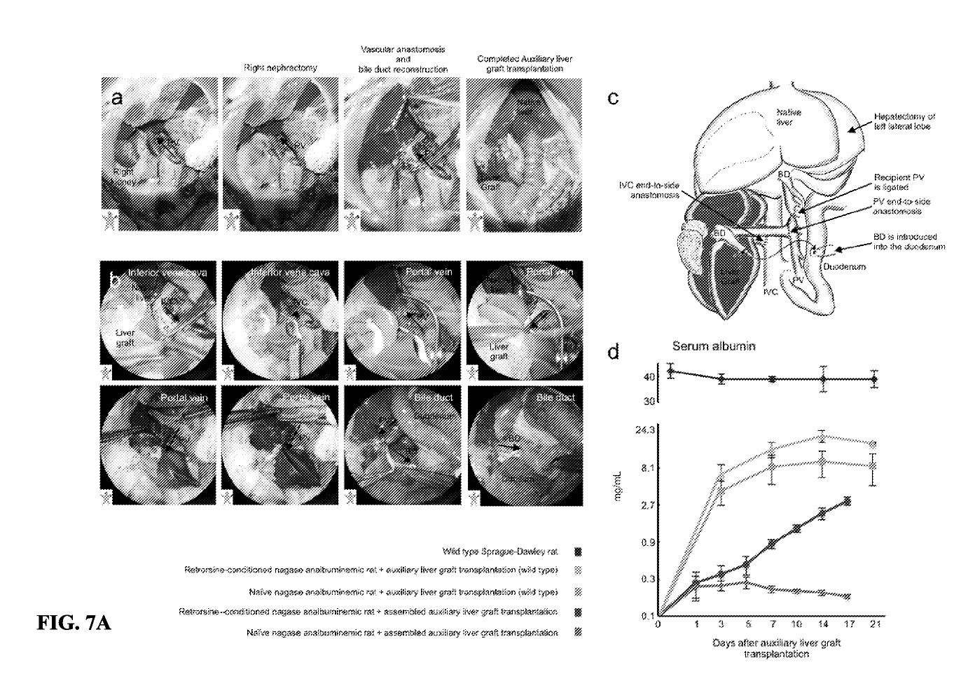

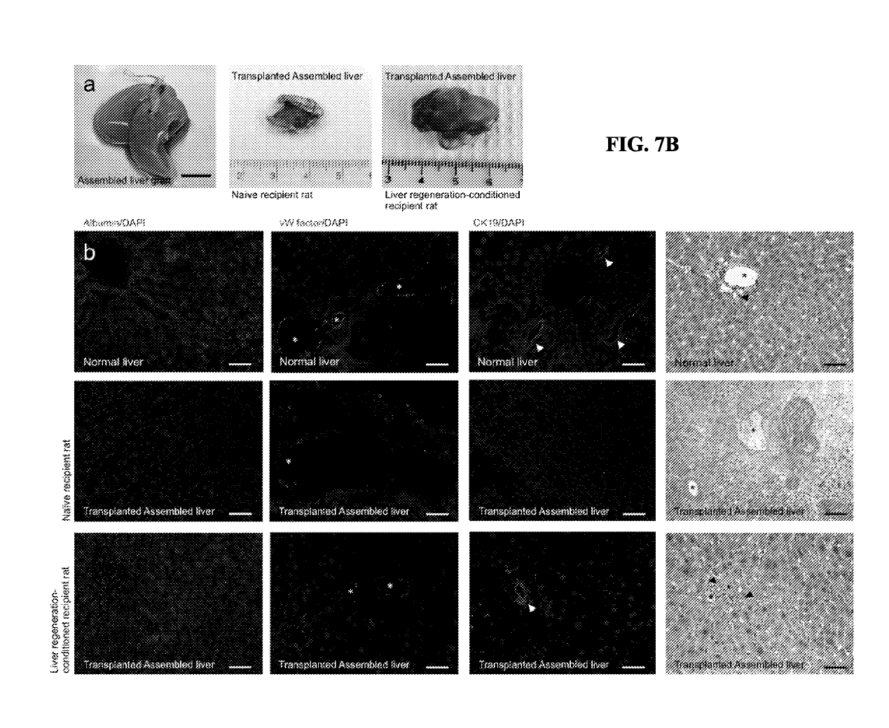

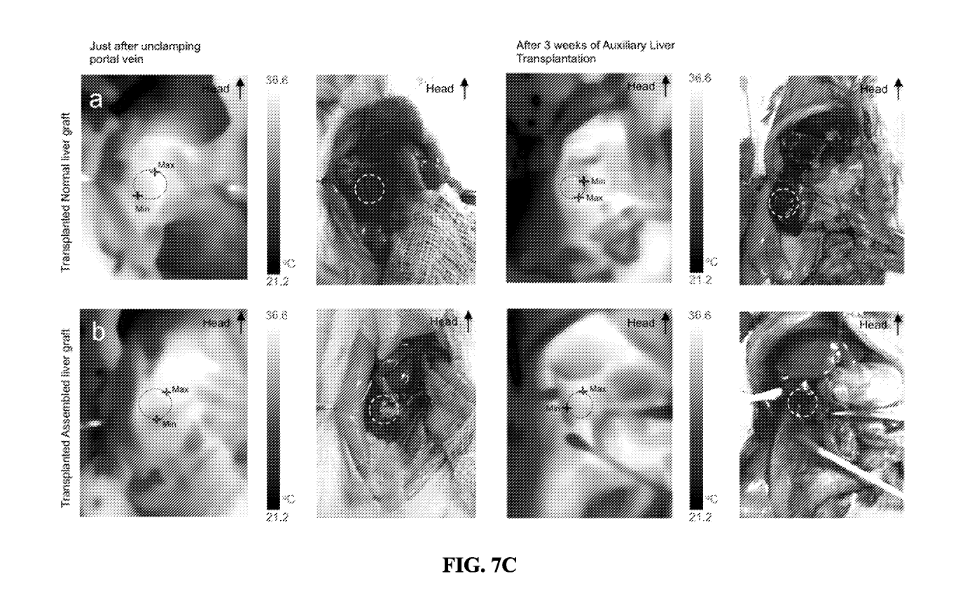

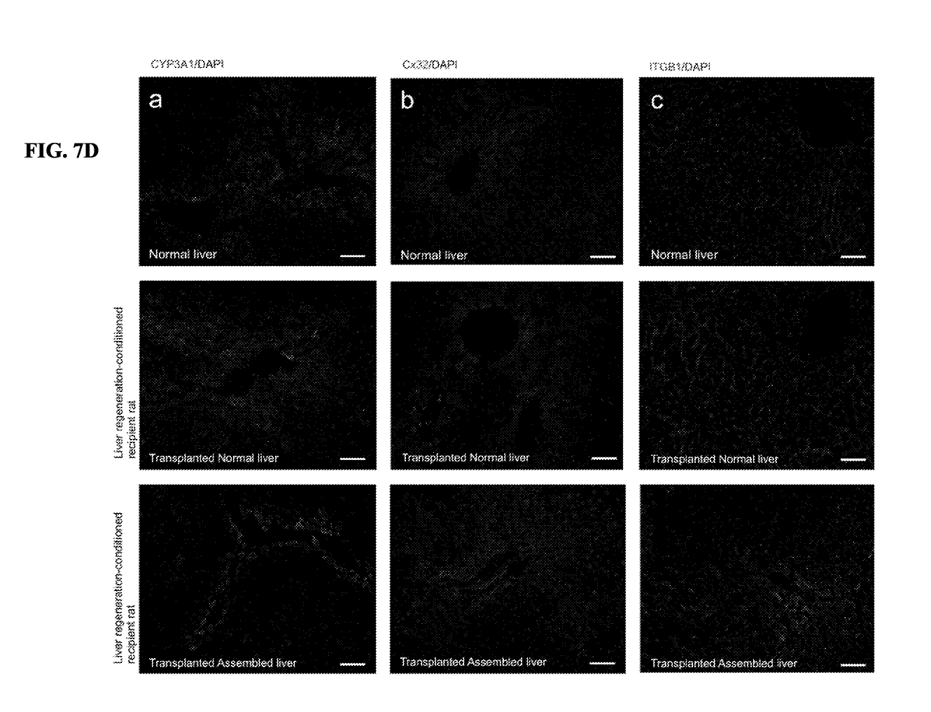

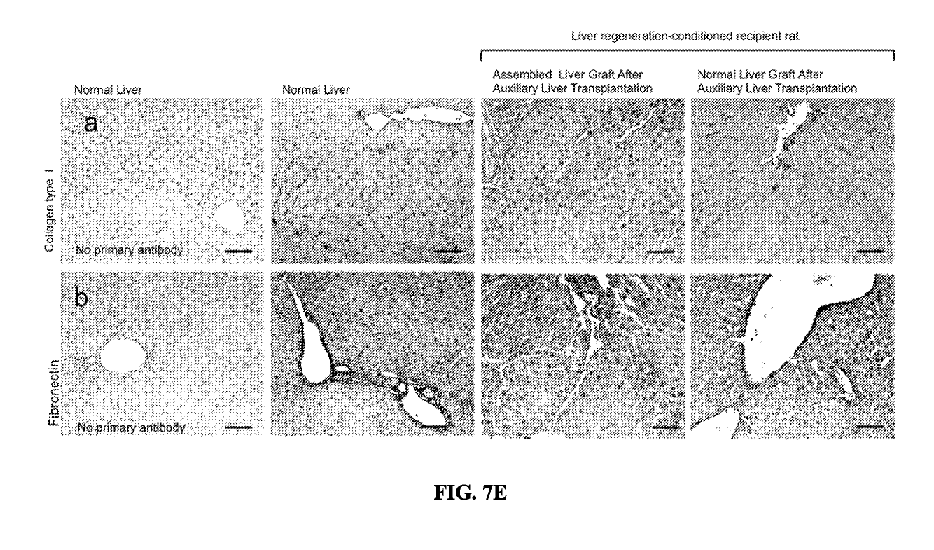

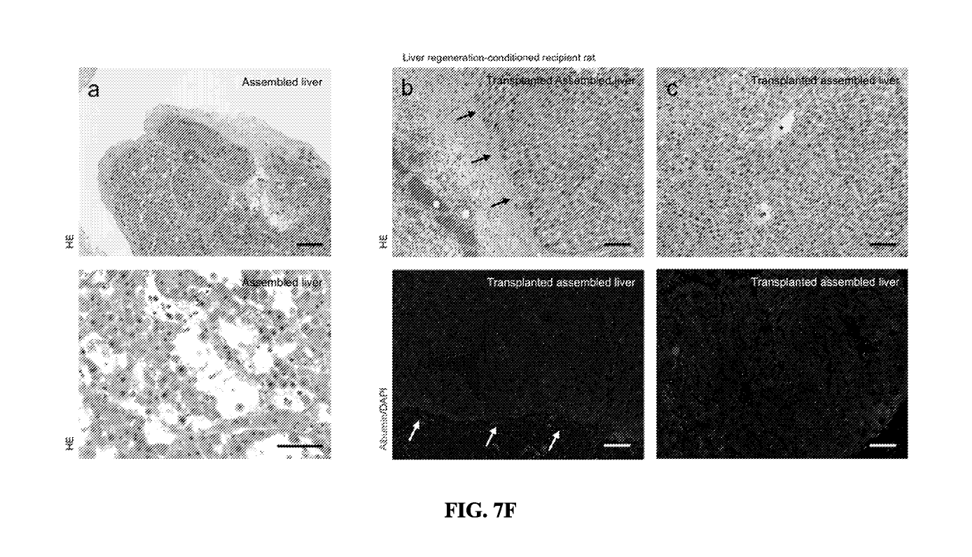

The next step was to develop an auxiliary liver transplant model in order to investigate engraftment, long-term function and the regenerative capacity of the assembled liver grafts (FIG. 7A, panels a-c). FIG. 7A, panel a shows representative images of graft transplantation; left to right: transplant site, transplant site after right nephrectomy, portal vein (PV) preparation for end-to-side anastomosis and auxiliary graft in contrast with the native liver. Panel (b) shows representative images of graft transplantation procedure; top, left to right: anterior wall of the infra-hepatic inferior vena cava (IVC) is cut and end-to-side anastomosis is performed, inferior vena cava blood flow is opened, PV is dissected and end-to-side anastomosis is performed; bottom, left to right: IVC and PV are de-clamped and the graft is re-perfused, PV is ligated above the anastomosis, bile duct (BD) of the graft is dissected and inserted into the duodenum. Panel (c) shows schematic representation of the auxiliary liver graft transplantation surgical technique for transplantation of normal and assembled liver grafts. Panel (d) shows blood albumin concentration of normal and assembled liver grafts in liver regeneration-conditioned (retrorsine-treated) (n=6, n=5) and naive (n=5, n=5) mutant nagase analbuminemic rats assayed by enzyme-linked immunosorbent assay (ELISA) error bars mean+s.e.m. Retrorsine-conditioned nagase analbuminemic rat+auxiliary liver graft transplantation versus naive nagase analbuminemic rat+auxiliary liver graft transplantation at 3 d (p=0.0305), 7 d (p=0.0044) and 14 d (p<0.0001), two-way ANOVA. conditioned nagase analbuminemic rat+assembled auxiliary liver graft transplantation versus naive nagase analbuminemic rat+assembled auxiliary liver graft transplantation at 3 d (p=0.9994), 7 d (p=0.9731), 14 d (p=0.7356), two-way ANOVA.

FIG. 7B, panel a shows representative photographs of gross morphology of an assembled liver graft before and after 17 d of auxiliary liver transplantation in naive and liver regeneration-conditioned (retrorsine-treated) mutant Nagase analbuminemic rats. Panel (b) shows immunohistochemical staining of assembled liver graft after 17 d of auxiliary liver transplantation (bottom two lines) compared to normal liver (top); left to right: albumin (red), Von Willebrand (vW) factor (red), Cytokeratin 19 (CK19) (red) and H&E. Arrows head point to bile duct structures in close proximity to vessels pointed by asterisk. Sections were counterstained with DAPI (blue). Scale bars: 50 μm (b).

FIG. 7C, panels a and b show (a) infrared image and corresponding photographs of normal and (b) assembled auxiliary liver grafts during transplantation and after 3 weeks of auxiliary liver transplantation. White/yellow areas indicate enhanced blood flow and black/purple areas indicate poor blood flow. Scale represents minimum and maximum temperature of circulated areas.

FIG. 7D shows histological analysis of transplanted normal and assembled liver grafts Immunohistochemical staining of normal and assembled liver graft after 14 d and 17 d of auxiliary liver transplantation respectively (bottom two lines) compared to normal liver (top); Panel (a): CYP3A1 (red), Panel (b) Conexxin-32 (Cx32) (red) (a key hepatic gap junction protein) and Panel (c) Integrin beta-1 (ITGB1) (red) (a key transmembrane receptor in the liver). Sections were counterstained with DAPI (blue). Scale bars: 50 μm (b).

FIG. 7E shows histological analysis of normal and assembled liver grafts after auxiliary liver transplantation Immunohistochemical staining of normal and assembled liver graft after 14 d and 17 d of auxiliary liver transplantation respectively compared to normal liver; Panel (a) Collagen type I; and Panel (b) Fibronectin. Sections were counterstained with Eosin (blue). Scale bars 50 μm.

FIG. 7F shows histological analysis of assembled liver graft before and after auxiliary liver transplantation. Panel (a) shows H&E staining of assembled liver graft before transplantation, showing a low and high magnification of the parenchyma space. Panels (b, c) show H&E and albumin (red) staining of assembled liver graft 17 d after transplantation in liver regeneration-conditioned (retrorsine-treated) mutant Nagase analbuminemic rats. Arrows point to the edge of an area of normal liver tissue seemingly constricted by the surrounding fibrotic tissue. Asterisk point to vessels in the liver tissue. Sections were counterstained with DAPI (blue). Scale bars (a) 200 μm (top) and 50 μm (bottom), (b) 100 μm.

As described, Nagase analbuminemic rats (NARs) were preconditioned by retrorsine treatment before transplantation in some studies to impair host hepatocyte replication capacity, allowing a regenerative advantage to the donor liver graft. To assess function and an increase in the mass of donor hepatocytes in the transplants, serum albumin was serially measured after transplantation (FIG. 7A, panel d). Since Nagase rats secrete no albumin, all measured albumin is generated from the auxiliary transplant. Shortly after assembling liver grafts with anti-thrombotic activity, a right nephrectomy was performed to create space for the donor liver graft and an end-side anastomosis was performed between donor and recipient portal vein and inferior vena cava. The graft stented bile duct was inserted to the recipient duodenum (FIG. 7A, panels a-c). Histological analysis of the assembled liver graft before and after transplantation is shown in FIG. 7F.

Prior to APLT, the recipient animal was injected with retrorsine and underwent a reduction of portal blood flow at the time of APLT, to create an environment where there was a selective growth advantage to transplanted grafts. The auxiliary partial graft was obtained by resection of the donor median and left lateral lobes, and was heterotopically transplanted into the recipient. Portal-portal anastomosis and infrahepatic-infrahepatic vena cava anastomosis were performed in an end-to-side manner and bile duct was implanted into the duodenum of the recipient. Graft survival was evaluated over time (up to 28 days) by graft weight, histological evaluation of proliferative markers and serum albumin levels in analbuminemic rats. FK506-based immunosuppression protocol effectively control graft rejection. Transplanted grafts revealed regenerative potential as evaluated by increase of liver mass weight of the donor graft. Serum albumin levels were maintained for the duration of the study. A novel auxiliary partial liver transplantation in rats for the future evaluation of engineered liver grafts was thus developed and standardized (FIG. 7A, panels a-c).

The regenerative effect of retrorsine preconditioning was not evident for the first days after auxiliary liver transplant. However, serum albumin levels increased continuously in retrorsine-conditioned recipient rats, reaching levels of 3.04±0.36 mg/mL on day 17 after transplantation, whereas, and levels in naive rats were 0.18±0.11 mg/mL. In contrast serum albumin levels were 16.71±0.60 mg/mL in retrorsine-conditioned recipient rats transplanted with normal liver grafts. Thus, the lower but parallel upward trend of serum albumin levels in assembled liver-transplanted conditioned-NAR recipients was approximately 18% that in animals transplanted with normal liver grafts. These results suggest that assembled liver grafts while functionally inferior to transplanted normal liver grafts (approximately one-fifth), demonstrated a proliferative/regenerative response when transplanted into animals preconditioned to deliver a regenerative stimulus to the graft (FIG. 7A, panel d).

The highest serum albumin levels in retrorsine-conditioned recipient rats transplanted with normal liver grafts were observed around 14-17 days after transplant, thus, further histological analysis was performed at this time. At seventeen days after transplantation into retrorsine conditioned recipients the diameter of assembled liver grafts measured from 2-3 cm and had the color and texture of a normal liver (FIG. 7B, panel a). In contrast, the assembled liver grafts recovered from recipients who were not conditioned with retrorsine measured less than half the size, at 1-1.5 cm, and appeared fibrotic and atrophic (FIG. 7B, panel a). Infrared thermal imaging demonstrated that blood flow was present at the moment of implantation and was maintained until the termination of the studies (FIG. 7C, panels a, b). Histological analysis of grafts from retrorsine-conditioned recipient rats demonstrated a tissue organization resembling normal liver with a nodular growth pattern (FIG. 7B, panel b; FIG. 6C, panels a-c) and displayed the classical cord arrangement. Albumin staining confirmed hepatic synthetic function in assembled grafts. Functional vessels were observed throughout the transplanted assembled liver grafts as demonstrated by the expression of Von Willebrand factor in endothelial cells. Present also were well formed, but scattered bile ducts, almost always near blood vessels, that stained positive for CK19 (FIG. 7B, panel b). However, the transplanted grafts showed little evidence of the classic portal triad-central vein relationship. In grafts recovered from unconditioned recipients, only dispersed albumin positive cells were found. Additional characterization, revealed that the zonated CYP3A1 expression followed the expected downstream perivenous region with a distribution of CYP3A1 restricted to a thin rim of one-three hepatocytes surrounding terminal hepatic venules in the transplanted assembled livers grafts from conditioned recipients (FIG. 7D, panel a). Cell-cell and cell-ECM interactions were reestablished as evidenced by the expression of Conexin-32 present throughout the liver tissue and Integrin β1, which showed areas of augmented expression throughout the liver tissue in the transplanted assembled liver grafts (FIG. 7D, panels b, c). Notably, ECM proteins (Collagen type I and Fibronectin) within the tissue of the transplanted assembled and normal liver grafts from conditioned recipients were remodeled and highly expressed as expected concurrent with regeneration (FIG. 7E, panels a, b). There was no apparent histological difference in native livers of the recipients NARs in all the experimental groups (naive and liver regeneration-conditioned rat) (data not shown).

Modification of Whole Liver Vascular Surface to Prevent Acute Thrombosis

The objective here was to achieve interruption of acute thrombosis in polyethylene-glycol-modified vascular surface of engineered liver grafts after re-connection to portal vein blood flow. It was previously demonstrated that modifying an injured vascular surface with a protein-reactive polymer could block undesirable platelet deposition (J Biomed Mater Res. 1998,41(2):251-6; J Vasc Surg. 2012, 55(4):1087-95). For this purpose, the utility of surface modification using a protein-reactive polymer, Nhydroxysuccinimide-polyethylene glycol, NHS-PEG to block platelet activation, deposition and formation of thrombus were evaluated. The entire vascular surfaces of the decellularized livers were coated, as indicated below (FIG. 6B) in combination with protocols of vascular endothelialization (FIG. 6B). Thus, decellularized livers were treated with NHS-PEG to modify their vascular surface and test the ability of biomaterials to block platelet deposition and thrombus formation. Briefly, PBS was added to lyophilized NHS-PEG (NANOCS, mPEG-NHS, PEG succinimidyl ester, MW 5000) to make 10 mL solution, and the solution was infused through the portal vein at a rate of 1-2 ml/min until the decellularized liver was completely filled, for a period of 20-30 minutes at room temperature. Different concentrations of PEG-NHS were tested (10 mg/ml, 30 mg/ml, 50 mg/ml, 100 mg/ml, 500 mg/ml, 1000 mg/ml) and histological evaluation of surface area coated using a PEG-NHS-biotin was carried out (FIG. 6D, panels a, b). FIG. 6D, panels a and b show (a) decellularized liver matrix treated with different doses of NHS-PEG-biotin and histological quantification of vessels covered with NHS-PEG-biotin (*p<0.0001 by one-way ANOVA, Turkey-Kramer) and (b) representative photographs of NHS-PEG treated decellularized livers and directly perfused with portal blood flow. FIG. 6D, panel c shows immunohistochemical staining for CD41 (platelet marker) and H&E staining of control and NHS-PEG treated decellularized liver matrix after perfusion of portal blood flow. Quantification of CD41 positive areas is also shown (p<0.0001 by Student's t-test). All error bars represent s.e.m. Scale bars (a,c) 100 μm.

Additionally, thrombus formation was quantified. Briefly, as described above the ECM-surface was modified with N-hydroxysuccinimide-polyethylene glycol (NHS-PEG) and was conjugated with biotin for detection purposes. 50 mg/mL of NHS-PEG-biotin cover 73±8% of the decellularized liver surface area. The ability of the coating to limit thrombosis was then tested by perfusion of coated livers with blood for approximately 15 min directly through the portal vein. PEG-NHS coated decellularized livers were reconnected to the blood flow by portal-portal anastomosis. Platelet deposition and thrombus formation was analyzed at several early time points (t=0, t=5, t=10, t=15, t=20, t=30). Thrombus formation was evaluated by: i) immunohistochemical analysis of CD41, ii) scanning electron microscope for platelet deposition and iii) measurement of blood pressure of the portal-portal anastomosis. There was a significant reduction in thrombus formation in the perfused NHS-PEG-coated decellularized livers (FIG. 6D, panels a-c). The important benchmark here is the degree of thrombosis blockage obtained by the use of protein-reactive polymer NHS-PEG in decellularized livers.

Assembling Liver Grafts for Transplantation

As described above, it was demonstrated that hepatocytes, endothelial and bile duct epithelial cells can be seeded into the whole-liver scaffolds and kept viable while providing essential liver functions. It was also demonstrated that acute thrombosis of decellularized whole livers after transplantation can be attenuated with re-endothelialization and vascular surface modification using protein-reactive polymers. Additionally, a clinically relevant rat model of auxiliary liver transplantation was described. Taken together, all this data demonstrated that re-cellularization protocols are compatible and can be performed efficiently while minimizing damage. Thus, the next step was to design the methods to engineer functional liver grafts and demonstrate long-term survival after transplantation.

Following the above protocols in 5 general steps produced transplantable liver grafts that survive for long-term (up to 17 days, at which time transplanted animals were sacrificed). The liver grafts transplanted in Retrorsine-treated Nagase rats demonstrated histological areas of liver sinusoidal tissue similar to normal liver. Histological tissue of the assembled and transplanted liver grafts was recovered after 3 and 17 days. H&E analysis demonstrated areas that showed liver tissue around the larger vessels, populating the surrounding parenchyma, and areas populated with inflammatory cells. These results demonstrate that the methods developed here are crucial to assembled liver grafts that achieve long term survival and function (17 days) compared to the previously published survival of assembled liver grafts (8 hours).

Scalability of Organ Decellularization Protocol

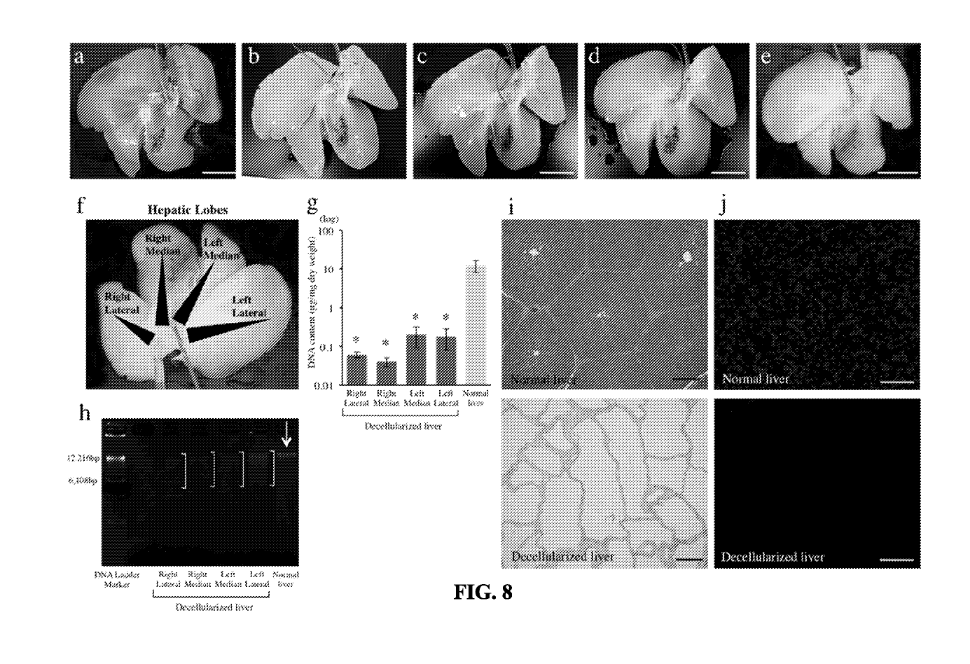

Scalability of Organ Decellularization Protocol. Different protocols for whole rat liver decellularization have been developed. To determine if the decellularization protocol was feasible in large livers, native whole porcine livers, which are similar to human in size and anatomy, were utilized. The decellularization protocol consisted first of a freezing-thawing technique for at least 12 hours to induce cellular lysis. The whole organ decellularization was achieved then by portal perfusion with sodium dodecyl sulfate (SDS), which is an anionic detergent that simultaneously can lyse cells and solubilize cytoplasmic components. The protocol was based on the rat liver decellularization protocol that was previously described above. Decellularization was achieved by perfusing the liver with sodium dodecyl sulfate (SDS; Sigma, St. Louis, MO, USA) in deionized water for a total of 72-96 h starting with 0.01% SDS for 24 h followed by 0.1% SDS for another 24 h, which was followed by 1% SDS for 48 h or more. Subsequently, the liver was washed with deionized water 15 min and with 1% Triton X-100 (Sigma) for 30 min. The decellularized livers were washed with PBS for 1 h. The liver bioscaffold was sterilized in 0.1% peracetic acid (Sigma) in PBS for 3 h. The liver bioscaffold was washed extensively with sterile PBS and preserved in PBS supplemented with antibiotics and kept at 4° C. for up to 7 days. (Yagi et al. Human-scale whole-organ bioengineering for liver transplantation: a regenerative medicine approach. Cell Transplant. 2013;22(2):231-42.) The objective of the studies below was to establish an effective and minimally disruptive method for the decellularization of intact porcine whole liver and to demonstrate that reconstitution of liver parenchyma is possible using the methodology developed in the rat model. Moreover, the bioreactors used to assemble whole livers were upscaled, and the anti-thrombotic studies previously developed in rodent studies were translated to the porcine model. The methods and techniques established in these studies represent a significant step towards the decellularization, re-cellularization and transplantation procedures necessary for a successful regenerative medicine approach to liver bioengineering for transplantation at a human scale. FIG. 8, panels a-e show macroscopic images of liver prior to decellularization and after various steps in the decellularization process. Representative images of porcine livers during decellularization process at (a) 0 h, (b) 18 h, (c) 48 h, (d) 72 h, and (e) 96 h. (f) DNA was extracted from each different lobe. (g) The DNA content of different lobes of the decellularized liver matrix (n=4 for each lobe) and (h) agarose gel electrophoresis of extracted DNA comparing to that of normal porcine liver. Histological comparison of normal liver and decellularized liver matrix: (i) hematoxylin and eosin. (j) The presence of intact nuclear material was evaluated by staining the decellularized liver and native liver using 4′,6-diamidino-2-phenylindole (DAPI). *p<0.01. Scale bars: 5 cm (a-e) and 100 μm (h, i). This protocol could create an acellular scaffold of porcine liver, which retains the gross shape of the whole organ.

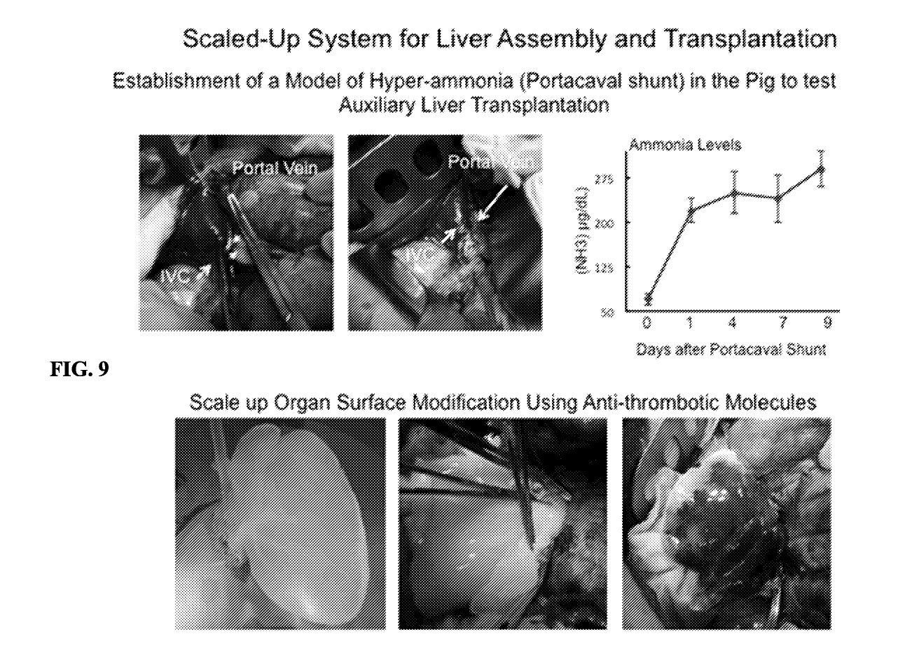

Immunological reaction of the remaining materials of the decellularized liver matrix has to be avoided if further clinical application is intended in order to elude any inflammatory reactions. As porcine livers have a much larger tissue density and area, the DNA content of the different areas and lobes was analyzed in order to measure the homogeneity of the decellularization process. Samples involved the right lateral, right median, left median and left lateral lobe of the decellularized whole liver (FIG. 8, panel 1). DNA content was decreased from 98.8±0.8% in all the liver lobes; 0.06±0.01 μg/mg dry weight (right lateral), 0.04±0.01 μg/mg dry weight (right median), 0.2±0.03 μg/mg dry weight (left median) and 0.18±0.04 μg/mg dry weight (left lateral), when compared to normal liver (12.12±0.8 μg/mg dry weight) (FIG. 9, panel g) indicating significant reduction of nuclear material of the whole liver. Extracted DNA was quantified by agarose gel electrophoresis, which showed smearing of the fragmented DNA bands from decellularized liver samples (FIG. 8, panel h). Histologic analysis with H&E stain (FIG. 8, panel i) and with DAPI (FIG. 8, panel j) showed no visible nuclear material in the decellularized liver matrix. This work demonstrates that the decellularization techniques developed in rodents can be scaled-up in large animals' livers.

A customized organ culture chamber, which was specifically constructed for a large-scale organ perfusion was developed; the perfusion system was designed based on previously developed system for rat liver that consisted of a peristaltic pump, bubble trap, and oxygenator. The system was placed in an incubator for temperature control, and the oxygenator was connected to atmospheric gas mixture. The graft was continuously perfused through the portal vein at 4 ml/min with continuous oxygenation that delivered an inflow partial oxygen tension of ˜300 mmHg.

### SUMMARY

The experiments and analyses above show:

i) Establishment of easy-to-use systems to monitor qualitatively the organ decellularization process based on a) multiphoton fluorescence microscopy, b) differential scanning calorimeter (DSC) analysis, c) DNA content and d) histological analysis of structural and basement membrane components (fibronectin and laminin);

ii) Optimized re-cellularization protocols for the vascular system (portal vein, central vein) and characterization of the functionality of the engineered liver vasculature based on a) histological evaluation, b) Ac-LDL incorporation, c) tPA reactive secretion and d) gene expression;

iii) Establishment of optimized re-cellularization protocols that combine three different compartments a) hepatocytes, b) microvascular endothelial cells and c) bile duct cells. Hepatic functionality using liver grafts re-cellularized with three different cell types is also reported;

iv) Design of protocols for the re-cellularization of the bile duct system and histological evaluation revealed that up to 60-70% of the bile ducts in the decellularized liver can be adequately re-cellularized with biliary epithelial cells;

v) Establishment and standardization of a clinically relevant model of Auxiliary Partial Liver Transplantation in the rat. This model represents a driving force of the laboratory as optimized protocols of liver engineering can easily be tested and validated. Immune-suppressed Nagase rats (analbuminemic rats) can be used, and serum albumin levels evaluated by ELISA to monitor the function of the transplanted graft show that the engineered tissue prepared according to the above provide such functionality;

vi) Development of an engineered liver graft with anti-thrombotic activity to achieve long-term survival after transplantation using optimized protocols to reconstitute the liver parenchyma and vascular endothelialization and polymer-based vascular surface modification to block acute thrombosis; and

vii) Establishment of scaled-up methods and techniques in porcine livers based on the systems developed in the rodent models (FIG. 9). The figure shows photographs (superior left) of a porta-caval shunt technique. Ammonia levels increased over time as shown in the graph. This model aids the testing of functionality of auxiliary liver transplantation. Representative photographs of decellularized livers directly perfused with portal blood flow (center bottom) in pigs to test molecules for anticoagulation according to one embodiment of a liver transplantation model using and testing the methods and organ structures described herein.

The present invention has been described with reference to certain exemplary embodiments, dispersible compositions and uses thereof However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of preparing a whole or partial organ extracellular matrix (ECM) construct comprising:
decellularizing a whole organ or partial organ by contacting the whole organ or partial organ with a decellularization solution; and
coating the decellularized whole organ or partial organ with an anticoagulant protein-associating composition.

2. The method of claim 1, in which the whole organ or partial organ is a whole liver or partial liver.

3. The method of claim 1, in which the step of decellularizing comprises contacting the whole organ or partial organ with a solution comprising about 0.02% trypsin and then contacting the whole organ or partial organ with a solution comprising about 0.1% Triton X-100.

4. The method of any of claim 1, in which the whole organ or partial organ is disinfected.

5. The method of claim 4, in which the whole organ or partial organ is disinfected with peracetic acid.

6. The method of any of claim 1, in which the decellularization solution further comprises a chelating agent.

7. The method of claim 6, in which the chelating agent is EGTA.

8. The method of claim 1, in which the whole organ or partial organ is frozen before decellularization.

9. The method of claim 1, in which the anticoagulant protein-associating composition comprises a polyether polymer, copolymer, or block copolymer, such as a poly(C₁-C₆ alkylene oxide) moiety, such as a polyoxyethylene, a polyoxypropylene, or a polyoxytetramethylene linked to an amine or ECM-reactive group.

10. The method of claim 1, in which the anticoagulant protein-associating composition comprises an N-hydroxysuccinimide (NHS) moiety covalently linked to a non-reactive, hydrophilic, biocompatible polymer moiety.

11. The method of claim 10, in which the biocompatible polymer moiety comprises a polyether polymer, copolymer, or block copolymer, such as a poly(C₁-C₆ alkylene oxide) moiety, such as a polyoxyethylene, a polyoxypropylene, or a polyoxytetramethylene linked to an amine-reactive group.

12. The method of claim 1, in which the anticoagulant protein-associating composition comprises poly(ethylene glycol) covalently linked to an NHS moiety.

13. The method of claim 1, in which the protein-associating polymer composition comprises a phosphorylcholine (PC), sulfobetaine (SB), or carboxybetaine (CB) moiety.

14. The method of claim 1, in which the anticoagulant protein-associating composition comprises an amine or ECM-reactive group.

15. The method of claim 14, in which the amine or ECM-reactive group is NHS, isocyanate (NCO), or carboxyl (COOH).

16. The method of claim 1, in which the anticoagulant protein-associating composition comprises one or more of PEG-NHS, PEG-NCO, PC-NHS, PC-NCO, SB-PEG-NHS, PC-COOH, SB-COOH, or poly[N-p-vinylbenzyl-4-O-β-D-galactopyranosyl-D-gluconamide]-co-valine (PVLA-co-VAL).

17. The method of claim 1, in which each of the decellularization solution and the anticoagulant protein-associating composition are provided to the whole organ or partial organ by flushing vasculature of the whole organ or partial organ, thereby coating the vasculature with the anticoagulant protein-associating composition.

18. A decellularized extracellular matrix (ECM) organ structure, comprising a decellularized whole organ or partial organ comprising native ECM structure, and an anticoagulant protein-associating composition dispersed within the native ECM structure.

19. The organ structure of claim 18, in which the anticoagulant protein-associating composition comprises one or more of PEG-NHS, PEG-NCO, PC-NHS, PC-NCO, SB-PEG-NHS, PC-COOH, SB-COOH, or PVLA-co-VAL.

20. The organ structure of claim 19, in which the anticoagulant protein-associating composition comprises poly(ethylene glycol) covalently linked to an NHS moiety

21. The organ structure of claim 18, further comprising orthotopic, autologous, allogeneic or xenogeneic cells dispersed into the decellularized organ structure.

22. The organ structure of claim 21, in which the cells are primary cells, multipotent cells, or pluripotent cells.
